(19) United States
(12) Patent Application Publication
(10) Pub. No.: US 2021/0338955 A1
(43) Pub. Date: Nov. 4, 2021

(54) CATHETER INFLATABLE CUFF PRESSURE STABILIZER

(71) Applicant: Airway Medix S.A., Warszawa (PL)

(72) Inventors: Oron ZACHAR, Tel Aviv (IL); Yair RAMOT, Kfar Maas (IL); Eizik AMAR, Ashdod (IL)

(21) Appl. No.: 17/146,038

(22) Filed: Jan. 11, 2021

Related U.S. Application Data
(63) Continuation of application No. 16/078,545, filed on Aug. 21, 2018, now abandoned.
(60) Provisional application No. 62/305,567, filed on Mar. 9, 2016.
(60) Provisional application No. 62/402,024, filed on Sep. 30, 2016.
(60) Provisional application No. 62/405,115, filed on Oct. 6, 2016.
(60) Provisional application No. 62/448,254, filed on Jan. 19, 2017.

(51) Int. Cl. A61M 16/04; A61M 16/00; G01L 7/18

(52) U.S. Cl. CPC A61M 16/044; A61M 16/0438; A61M 16/0003; G01L 7/18; A61M 2205/3341; A61M 2205/332; A61M 2205/583; A61M 2205/273; A61M 2209/08; A61M 2205/3348; A61M 2205/215; A61M 2016/0027

(57) ABSTRACT

A cuff pressure stabilizer (100, 200, 300, 500, 600, 800) is provided that includes an inflation lumen proximal port connector (134), which is shaped to form an air-tight seal with an inflation lumen proximal port (15) of a catheter (10) additionally having an inflatable cuff (11) and an inflation lumen (13); a fluid reservoir (120, 524, 624); a liquid column container (118, 518, 618), which is (a) open to the atmosphere (99) at at least one site along the liquid column container, (b) in fluid communication with the fluid reservoir (120, 524, 624), and (c) in communication with the inflation lumen proximal port connector (134) via the fluid reservoir (120, 524, 624); and a liquid (121), which is contained (a) in the fluid reservoir (120, 524, 624), (b) in the liquid column container (118, 518, 618), or (c) partially in the fluid reservoir (120, 524, 624) and partially in the liquid column container (118, 518, 618), and which has a density of between 1.5 and 5 g/cm3 at 4 degrees Celsius at 1 atm.

### CROSS-REFERENCE TO RELATED APPLICATIONS

The present patent application is a continuation of U.S. patent application Ser. No. 16/078,545 which is incorporated herein by reference. U.S. patent application Ser. No. 16/078,545 is a national-stage entry of PCT/IL2017/050284 filed on Mar. 8, 2017 which is incorporated herein by reference. PCT/IL2017/050284 claims priority from (a) U.S. Provisional Application 62/305,567, filed Mar. 9, 2016, (b) U.S. Provisional Application 62/402,024, filed Sep. 30, 2016, (c) U.S. Provisional Application 62/405,115, filed Oct. 6, 2016, and (d) U.S. Provisional Application 62/448,254, filed Jan. 19, 2017, all of which are assigned to the assignee of the present application and are incorporated herein by reference.

### FIELD OF THE APPLICATION

The present invention relates generally to medical suction catheter systems, and specifically to endotracheal tube inflatable cuff manometers.

### BACKGROUND OF THE APPLICATION

Suction catheters are commonly used to aspirate tracheobronchial fluids in patients ventilated with endotracheal tube (ETT) and tracheostomy tube devices.

UK Publication GB 2482618 A to Einav et al., which is assigned to the assignee of the present application and is incorporated herein by reference, describes a multi-lumen catheter for multiple fluids conduction, including balloon inflation with air via an inflation lumen, suction via a suction lumen, and cleaning fluids delivery via a cleaning fluid-delivery lumen.

U.S. Pat. No. 8,999,074 to Zachar et al., which is assigned to the assignee of the present application and is incorporated herein by reference, describes a cleaning catheter that includes fluid-delivery and suction lumens. A flow regulator defines suction and fluid ports. A mechanical user control element is configured to mechanically and non-electrically set activation states of the flow regulator, and transition between first and third configurations via a second configuration. When the control element is in the first configuration, the flow regulator blocks fluid communication (a) between the suction port and the suction lumen and (b) between the fluid port and the fluid-delivery lumen. When the control element is in the second configuration, the flow regulator effects fluid communication between the suction port and the suction lumen, and blocks fluid communication between the fluid port and the fluid-delivery lumen. When the control element is in the third configuration, the flow regulator effects fluid communication (a) between the suction port and the suction lumen and (b) between the fluid port and the fluid-delivery lumen.

Some ETTs comprise an inflatable cuff, which forms a seal against the tracheal wall. This seal prevents gases from leaking past the cuff and allows positive pressure ventilation. Desired safe inflatable cuff pressure is in the range of 23-27 cm H2O, with optimal pressure about 25 cm H2O. Pressure above 30 cm H2O can cause irritation to the surrounding tracheal tissue. Extended duration of such high cuff pressure can interfere with oxygen flow to the tissue, causing tissue necrosis and a substantial wound. Low cuff balloon pressure, typically below 20 cm H2O, compromises the cuff sealing performance, and allows leakage into the lungs of subglottic fluids descending from above the balloon.

The external surface of inflatable cuffs is in communication with the ventilation pressure of the lungs. The pressure of the inflatable cuff cycles with the ventilation cycle. When an artificially-ventilated patient is also anesthetized, the plastic of the inflatable cuff absorbs the nitrous oxide (N2O) gas used in anesthesia, which increases pressure in the cuff.

In current clinical settings of intensive care patients, changes of body positioning lead to significant changes in cuff pressure in the range of 10-50 cm H2O, i.e., out of the safe range of 20-30 cm H2O, and certainly out of the desired range of 23-27 cm H2O. See, for example, Lizy C et al., “Cuff pressure of endotracheal tubes after changes in body position in critically ill patients treated with mechanical ventilation,” Am J Crit Care. 2014 January; 23(1):e1-8.

Therefore, there is a need to safely maintain the inflatable cuff pressure is in the range of 23-27 cm H2O, optimally about 25 cm H2O, and to avoid extended periods of pressure above 30 cm H2O. In particular, there is a need to suppress the fluctuations of pressure in clinical settings caused by patient change of body positions.

Currently, the most common practiced approach for ETT cuff pressure management is manual monitoring (using a manometer) and adjustment of cuff pressure, which contributes to ICU staff workload. It has been shown that up to eight manual adjustments of cuff pressure are required daily to maintain recommended cuff pressure ranges. Even so, the cuff pressure is uncontrolled during the long time periods between manual cuff adjustments. In addition, the manometer must be connected to and disconnected from the ETT cuff for each pressure measurement, which allows a small amount of air to escape from the ETT cuff. Still further, many conventional ETT manometers lose calibration relatively quickly.

Prior art cuff pressure regulators can be divided into two groups: (a) large bedside non-disposable expensive electric pump and electronic pressure monitors; and (b) small and light disposable non-electric limited-pressure reservoir compartments that must be filled manually. Use of disposable devices both prevents cross-contamination between patients and obviates the need for costly sterilization processes between patients. Moreover, the compactness of the disposable devices allows them to be attached on the ETT circuit and not occupy bedside space and an electric power cable connection.

### SUMMARY OF THE APPLICATION

Some applications of the present invention provide a cuff pressure stabilizer for use with a gas and a catheter, such as a tracheal ventilation tube that comprises inflatable cuff, an inflation lumen, and an inflation lumen proximal port. The cuff pressure stabilizer is configured to provide automatic pressure regulation of the inflatable cuff, while simultaneously continuously displaying the pressure in the inflatable cuff.

In some applications of the present invention, the cuff pressure stabilizer comprises (a) an inflation lumen proximal port connector, which is shaped to form an air-tight seal with the inflation lumen proximal port of tracheal ventilation tube, (b) a fluid reservoir, (c) a liquid column container, which is (i) open to the atmosphere at at least one site along the liquid column container, (ii) in fluid communication with the fluid reservoir, and (iii) in communication with the inflation lumen proximal port connector via the fluid reservoir and (d) a liquid, which is contained in the fluid reservoir and/or in liquid column container. Typically, the liquid has a density of between 1.5 and 5 g/cm3 at 4 degrees Celsius at 1 atm, and/or a density of between 1.5 and 5 g/cm3 at 20 degrees Celsius at 1 atm.

When the system is in equilibrium, the pressure of the gas in the fluid reservoir equals the pressure of the gas in the inflatable cuff. The cuff pressure stabilizer typically provides uninterrupted fluid communication between the inflatable cuff and the fluid reservoir, including during pressure measurement. The cuff pressure stabilizer has a plurality of pressure indicia markings distributed along the liquid column container for measuring a height of the liquid in the liquid column container. The liquid column container is typically used in an aligned orientation in which the pressure indicia markings reflect, to within 1 cm H2O (i.e., with no error or an error of no more than 1 cm H2O), pressure of the gas in the fluid reservoir at least in a relevant pressure range of 23-27 cm H2O, such as a range of 22-28 cm H2O, e.g., a range of 20-30 cm H2O. The pressure is read by comparing a level of fluid in the liquid column container with the pressure indicia markings, as is known in the manometer art.

For some applications, when the liquid column container is oriented in the aligned orientation, the liquid column container has an inner cross-sectional area, measured in a horizontal plane, of at least 0.16 cm2 (e.g., at least 0.25 cm2, 0.5 cm2, or 1 cm2) at a plurality of (such as at most or all) axial locations along the liquid column container corresponding to the pressure of the gas in the fluid reservoir at a respective plurality of pressures in the relevant pressure range. As a result of this relatively large cross-sectional area, the cuff pressure stabilizer regulates (i.e., reduces fluctuations) the pressure of the gas in the fluid reservoir at least for changes of gas volume in the range of 0-2 cc, and thus at a gas inlet and in the inflatable cuff, in addition to measuring the pressure. In contrast, conventional manometers only measure the pressure, without substantially affecting the pressure, as it is ideally and commonly the goal of measurement devices to not affect the measured target. For a given cuff of initial gas volume V, as the squeezing of the inflatable cuff by the trachea increases such that the available gas volume decreases, the volume of the gas in the inflatable cuff decreases by some fraction equal to the change in V divided by V because the cuff is nearly non-compliant. For endotracheal tubes without external regulation, this decrease in volume of the inflatable cuff results in an increase in pressure of the gas within the system, including within the inflatable cuff, since the gas contained in the cuff has no significant external volume to move into, in accordance with the ideal gas law; the opposite occurs when the level of squeezing on the cuff by the trachea decreases.

In experiments conducted by the inventors, the inventors found that, for real endotracheal tube cuff balloons of volumes around 10 cc, each 0.1 cc decrease in volume in the inflatable cuff resulted in about a 1 cm H2O increase in pressure in the system and the cuff, and each 0.1 cc increase in volume in the inflatable cuff resulted in about a 1 cm H2O decrease in pressure in the system and the cuff. This is a surprising significant departure from the ideal non-compliant gas law calculation which would predict a 0.01 cc volume change per 1 cm H2O pressure change. The inventors thus concluded that real endotracheal tube cuff balloons are in fact semi-compliant. Therefore, the mitigation volumes should be calculated based on the experimental finding. In clinical practice, the pressure in ETT inflatable cuffs generally varies +/−10 cm H2O from the typically target pressure of 25 cm H2O, i.e., varies between 15 and 35 cm H2O. Based on the above-mentioned experimental data, the inventors appreciated that the volume in ETT inflatable cuffs generally varies by +/−1 cc (+/−10 cm H2O times 0.1 cc/cm H2O), i.e., a total range of 2 cc, and, among a broader spectrum of patients, the volume of ETT inflatable cuffs generally varies by +/−20 cm H2O from the typically target pressure of 25 cm H2O, i.e., a total range of at least 4 cc.

The inventors appreciated that to the extent that the cuff pressure stabilizer is able to offset the changes in volume in the inflatable cuff, the pressure changes are also offset, thereby stabilizing the pressure in the inflatable cuff. The cuff pressure stabilizer is able to offset the changes in volume in the inflatable cuff because of the relatively large cross-sectional area of the liquid column container at the relevant pressure range, e.g., 23-27 cm H2O.

For example, assume that (a) the liquid comprises water, (b) the cross-sectional area of the liquid column container at the relevant pressure range is 1 cm2, (c) the pressure indicia markings are spaced at 1-cm intervals, and (d) the initial pressure in the inflatable cuff is 25 cm H2O. A decrease in volume of the inflatable cuff of 1 cc (caused by increased squeezing by the trachea) would displace from the inflatable cuff the excess 1 cc of gas into the fluid reservoir, and a corresponding additional 1 cc of water out of the fluid reservoir into the liquid column container. This additional 1 cc of water would fill an additional 1 cc of the fluid reservoir, raising the level of fluid by 1 cm, and thus the pressure in the inflatable cuff (as indicated by the pressure indicia markings) by 1 cm H2O, from 25 cm H2O to 26 cm H2O.

For a real inflatable cuff having a volume of 10 cc without attachment of the regulation system, a decrease in volume of the inflatable cuff of 1 cc would have resulted in an increase of the cuff pressure gas by about 10 cm H2O, based on the inventors' experimental data, i.e., the integration of the pressure regulator with the tracheal ventilation tube results in a factor of 10 suppression of the pressure change, resulting in the pressure regulation described herein.

Typically, the liquid column container is wider (i.e., has a greater cross-sectional area) in (a) a relevant-range fluid compartment that includes the relevant pressure range than in (b) a lower-range fluid compartment that reflects pressures of the gas in the fluid reservoir of less than 20 cm H2O. The narrower liquid column container in the lower-range fluid compartment reduces the total required amount of the liquid, which is useful in configurations in which the liquid comprises an expensive heavy liquid.

For some applications, the liquid column container is wider (i.e., has a greater cross-sectional area) in (a) a buffer fluid compartment that reflects pressures of the gas in the fluid reservoir of greater than 28 cm H2O, e.g., greater than 30 cm H2O that includes the relevant pressure range than in (b) the relevant-range fluid compartment that includes the relevant pressure range. The wider liquid column container in the buffer fluid compartment substantially reduces increases in pressure if the pressure should exceed the lower end of the pressure range of the buffer fluid compartment, because the buffer fluid compartment can hold a greater volume of liquid per unit of height than can the relevant-range fluid compartment.

For some applications, the liquid has a density of between 0.8 and 12 g/cm3 at 4 degrees Celsius at 1 atm, and/or a density of between 0.8 and 12 g/cm3 at 20 degrees Celsius at 1 atm. Typically, the density (whether at 4 degrees or at 20 degrees) is between 1.5 and 5 g/cm3, such as between 2 and 4 g/cm3, e.g., between 2.5 and 3.5 g/cm3 (all of these values are more dense than water and less dense than mercury). For some applications, the liquid is selected from the group consisting of: sodium polytungstate, sodium metatungstate, lithium polytungstate, and lithium metatungstate.

To the extent that the density of the liquid is greater than that of water, i.e., greater than 1 g/cm3 at 4 degrees Celsius at 1 atm, a shorter the liquid column container can be used to measure and regulate pressures, and the pressure indicia markings are closer together. Assuming a container of uniform cross section and a liquid column of uniform cross section, it follows that, if using a liquid of the density compared with using water, the distance between the pressure indicia markings for indicating a 1 cm H2O change in pressure equals the quotient of (a) 1 cm divided by (b) the density of the liquid at 4 degrees Celsius at 1 atm.

Typically, the liquid column container is open to the atmosphere at at least one site along the liquid column container. The liquid column container has first and second ends at opposite ends of the liquid column container. For some applications, the liquid column container is in fluid communication with the fluid reservoir via the first end, the at least one site is at the second end, and the liquid column container is open to the atmosphere at the second end. For some applications, the liquid column container defines an opening having an area of between 0.09 and 1 mm2, and the liquid column container is open to the atmosphere via the opening.

Typically, the volume of the liquid is less than the volume of the wider portion of the liquid column container in the relevant pressure range. Under normal working conditions, there is a fixed amount of gas enclosed between the cuff balloon and the liquid. As the pressure within the cuff changes, only the fraction of the gas is in the cuff and the fraction of the gas outside the cuff change. However, since the liquid total volume is less than the total volume of the gas in the cuff, there is an upper limit to the magnitude of volume variation in the cuff that can be offset by only movement of the liquid surface. Consequently, if the inflatable cuff is squeezed to a high threshold pressure at which all available liquid has moved into the wider portion of the liquid column container, further squeezing of the inflatable cuff releases gas bubbles through the liquid in the liquid column container and into fluid communication with the atmosphere, and thus prevents the pressure from further increasing within the inflatable cuff. If the volume of the liquid were instead greater than the volume of the wider portion of the liquid column container, some liquid would spill out of the liquid column container and prevent continued accurate function of the cuff pressure stabilizer. (A small amount of the liquid generally remains in the reservoir regardless of the pressure in the cuff because of the geometry of the interface between the reservoir and the liquid column container.) This arrangement effectively serves the same function as a maximum pressure release valve.

For some applications, the cuff pressure stabilizer further comprises (a) an inflation inlet port, which is coupleable with an external inflation source, such as a syringe, (b) a first connector tube, which couples the inflation lumen proximal port connector in fluid communication with the inflation inlet port, and (c) a second connector tube, which couples the gas inlet in fluid communication with the inflation inlet port, such that the inflation lumen proximal port connector is in fluid communication with the gas inlet via the first connector tube and the second connector tube. For some applications, the cuff pressure stabilizer further comprises an inlet junction, which comprises the inflation inlet port, and which couples in fluid communication the inflation inlet port, the first connector tube, and the second connector tube.

For some applications, when (a) the inflation lumen proximal port connector forms the air-tight seal with the inflation lumen proximal port of the tracheal ventilation tube and (b) a pressure of the gas of the gas in the fluid reservoir is 10 cm H2O, (i) a first combined air-flow resistance between the inflation inlet port and an interior of the inflatable cuff equals between 80% and 120% of (ii) a second combined air-flow resistance between inflation inlet port and the fluid reservoir, such as between 90% and 110%, e.g., between 95% and 105%. Typically, in order to achieve these relative air-flow resistances, the relative lengths of the first and second connector tubes are set such that the resistance of the second connector tube equals the sum of the resistance of the first connector tube and a fixed constant resistance of all elements of the tracheal ventilation tube in the flow path. This approximately equal air-flow resistance prevents transient false pressure readings immediately following inflation or reinflation of the inflatable cuff via the inflation inlet port, without being dependent on the technique of the healthcare worker. For example, if the resistance from the inflation inlet port were lower in the second connector tube (to the cuff pressure stabilizer) than in the first connector tube (to the inflatable cuff), during inflation initially a majority of the air would flow toward the cuff pressure stabilizer. As a result, the level of fluid in the liquid column container would indicate a higher pressure than the true pressure of the inflatable cuff. If the external inflation source were to be disconnected at this point in time, the pressure shown by the liquid column container would gradually decrease as pressure equilibrium between the inflatable cuff and the fluid reservoir is gradually reached.

For some applications, the cuff pressure stabilizer further comprises one or more connector tubes, which couple the inflation lumen proximal port connector in fluid communication with the gas inlet. When the inflation lumen proximal port connector forms the air-tight seal with the inflation lumen proximal port of the tracheal ventilation tube, a combined air-flow resistance between an interior of the inflatable cuff and the fluid reservoir is such that a transient pressure difference of 5 cm H2O between the interior of the inflatable cuff and the fluid reservoir results in less than a 0.05, a 0.02, or a 0.01 cc per second, fluid flow from the inflatable cuff into the fluid reservoir. The slow rate of flow delays the automatic pressure-regulation response from the cuff pressure stabilizer. A too rapid pressure-regulation response might underinflate the inflatable cuff during transient, short-term increases in pressure in the inflatable cuff, such as during the positive pressure phase of the ventilation cycle when high-pressure ventilation (generally greater than 25 cm H2O) is applied to the patient, generally for only a few seconds, typically less than 3 seconds.

For some applications, the liquid column container is arranged such that, when (a) the liquid column container is oriented in the aligned orientation and (b) the pressure of the gas in the fluid reservoir is 25 cm H2O: an increase in a volume of the gas in the fluid reservoir of up to 2 cc results in less than a 10 cm H2O increase in the pressure of the gas in the fluid reservoir, such as less than a 6 cm H2O increase in the pressure of the gas in the fluid reservoir, e.g., less than a 5 cm H2O or less than a 4 cm H2O increase.

Typically, the cuff pressure stabilizer does not comprise any membranes in contact with the liquid, and does not comprise any membranes in a fluid path between the liquid and the atmosphere.

In some applications of the present invention, the liquid column container is shaped so as to define a wider portion and a narrower portion axially between the wider portion and the fluid reservoir, and the cuff pressure stabilizer is arranged to provide an adjustable distance between a wider portion of the liquid column container and the fluid reservoir. Providing the adjustable difference allows a healthcare worker to optimize the cuff pressure stabilizer for a desired target pressure and target pressure range. For example, some patients that are ventilated at higher pressure need higher average cuff pressures, e.g., 28 cm H2O or 30 cm H2O, rather than the typical 25 cm H2O target. There is also a range of preferred target pressures even for patients ventilated at normal pressure, depending on the patient's particular circumstances and the medical opinion of the physician.

For some applications, the wider portion of the liquid column container has a target-pressure indicator marker, which is axially slidable with respect to the pressure indicia markings, which are typically provided on the casing. The healthcare worker may set the target-pressure indicator marker to indicate a desired target pressure in the inflatable cuff, and then inflate the inflatable cuff at least approximately to this target pressure. Such setting of the target-pressure indicator marker, by axially moving the target-pressure indicator marker with respect to the pressure indicia markings, has the effect of adjusting the adjustable distance between the wider portion of the liquid column container and the fluid reservoir. For some applications, at least an axial portion of the narrower portion of the liquid column container is flexible, so as to provide a variable axial length to the narrower portion.

For some applications, the narrower portion of the liquid column container is generally thin and flat, for example, shaped generally as a ribbon. For example, at each of all axial locations along the narrower portion of the liquid column container, the liquid column container may (a) have a largest inner dimension equal to a greatest distance between any two points within the liquid column container in (i) the above-mentioned horizontal plane, and/or (ii) a plane perpendicular to the longitudinal axis of the liquid column container, and (b) be able to encompass a largest circle in the horizontal plane. At most or all (e.g., all) of the axial locations along the narrower portion of the liquid column container, a ratio of (a) the largest inner dimension to (b) the diameter of the circle equals at least 2:1, such as at least 4:1, e.g., at least 8:1. For some applications, at most or all of the axial locations along the narrower portion of the liquid column container, the liquid column container has a non-circular cross-sectional shape, such as a rectangle, an oblong shape, an ellipse, or a crescent. Alternatively or additionally, for some applications, at each of the axial locations along the narrower portion of the liquid column container, at least 80% of the inner cross-sectional area is within 1 mm of an inner surface of the liquid column container, such as within 0.5 mm of the inner surface.

The relatively thin flat shape of the cross-sectional shape of the narrower portion of the liquid column container prevents gas bubbles from occluding the narrower portion. The surface tension of the bubble causes the bubble not to reach the edges of the liquid column container. By contrast, in configurations in which the narrower portion is circular in cross-section, gas bubbles may sometimes occlude the narrower portion, particularly if the diameter of the tube is very small. The generally thin flat shape of the cross-sectional shape generally prevents such occlusion.

There is therefore provided, in accordance with an inventive concept 1 of the present invention, apparatus for use in contact with the atmosphere of the Earth and for use with a gas and a catheter having an inflatable cuff, an inflation lumen, and an inflation lumen proximal port, the apparatus including a cuff pressure stabilizer, which includes:

an inflation lumen proximal port connector, which is shaped to form an air-tight seal with the inflation lumen proximal port of the catheter;

a fluid reservoir;

a liquid column container, which is (a) open to the atmosphere at at least one site along the liquid column container, (b) in fluid communication with the fluid reservoir, and (c) in communication with the inflation lumen proximal port connector via the fluid reservoir; and

a liquid, which is contained (a) in the fluid reservoir, (b) in the liquid column container, or (c) partially in the fluid reservoir and partially in the liquid column container, and which has a density of between 1.5 and 5 g/cm3 at 4 degrees Celsius at 1 atm,

wherein the cuff pressure stabilizer has a plurality of pressure indicia markings distributed along the liquid column container tube for measuring a height of the liquid in the liquid column container.
Inventive concept 2. The apparatus according to inventive concept 1, wherein the density of the liquid is less than 3.5 g at 4 degrees Celsius at 1 atm.
Inventive concept 3. The apparatus according to inventive concept 2, wherein the density of the liquid is less than 3 g at 4 degrees Celsius at 1 atm.
Inventive concept 4. The apparatus according to inventive concept 1, wherein the liquid column container is in pressure communication with the inflation lumen proximal port connector via the fluid reservoir.
Inventive concept 5. The apparatus according to inventive concept 4, wherein the fluid reservoir includes at least one wall that includes a pressure-communicating movable wall, and wherein the liquid column container is in pressure communication with the inflation lumen proximal port connector via the pressure-communicating movable wall of the fluid reservoir.
Inventive concept 6. The apparatus according to inventive concept 5, wherein the pressure-communicating movable wall includes a flexible membrane.
Inventive concept 7. The apparatus according to inventive concept 1, wherein the liquid has a viscosity of no more than 25 times a viscosity of water at 4 degrees Celsius at 1 atm.
Inventive concept 8. The apparatus according to inventive concept 7, wherein the viscosity of the liquid is no more than 10 times the viscosity of water at 4 degrees Celsius at 1 atm.
Inventive concept 9. The apparatus according to inventive concept 1, wherein the liquid includes a solution of crystals dissolved in a liquid solvent having a mass percent of between 65% and 85%.
Inventive concept 10. The apparatus according to inventive concept 1, wherein the liquid is non-toxic.
Inventive concept 11. The apparatus according to inventive concept 1, wherein the liquid is non-flammable.
Inventive concept 12. The apparatus according to inventive concept 1, wherein the liquid is odorless.
Inventive concept 13. The apparatus according to inventive concept 1, wherein the liquid includes a tungstate-based liquid.
Inventive concept 14. The apparatus according to inventive concept 13, wherein the liquid is selected from the group consisting of: sodium polytungstate, sodium metatungstate, lithium polytungstate, and lithium metatungstate.
Inventive concept 15. The apparatus according to inventive concept 14, wherein the liquid includes sodium polytungstate.
Inventive concept 16. The apparatus according to inventive concept 14, wherein the liquid includes sodium metatungstate.
Inventive concept 17. The apparatus according to inventive concept 14, wherein the liquid includes lithium polytungstate.
Inventive concept 18. The apparatus according to inventive concept 14, wherein the liquid includes lithium metatungstate.
Inventive concept 19. The apparatus according to inventive concept 1, wherein the liquid includes at least two liquids, at least one of which has the density of between 1.5 and 5 g/cm3 at 4 degrees Celsius at 1 atm, and at least one of which has a density of less than 1.5 g/cm3 at 4 degrees Celsius at 1 atm.
Inventive concept 20. The apparatus according to inventive concept 1, wherein the cuff pressure stabilizer includes a gas container, which:

extends to the inflation lumen proximal port connector,

contains some of the gas,

is not in liquid communication with the fluid reservoir, and

includes at least one wall that includes a volume-compensation movable wall, which is in pressure communication with the atmosphere.
Inventive concept 21. The apparatus according to inventive concept 20, wherein the volume-compensation movable wall includes a flexible membrane.
Inventive concept 22. The apparatus according to inventive concept 20, wherein the fluid reservoir includes at least one wall that includes a pressure-communicating movable wall, and wherein the liquid column container is in pressure communication with the gas container via the pressure-communicating movable wall of the fluid reservoir.
Inventive concept 23. The apparatus according to inventive concept 22, wherein the pressure-communicating movable wall includes a flexible membrane.
Inventive concept 24. The apparatus according to inventive concept 22, wherein the pressure-communicating movable wall is disposed at least partially within the gas container.
Inventive concept 25. The apparatus according to inventive concept 22,

wherein the gas container includes a buffer chamber, which is shaped so as to define a chamber inlet port that is in fluid communication with the inflation lumen proximal port connector, and

wherein the pressure-communicating movable wall is disposed at least partially within the buffer chamber.
Inventive concept 26. The apparatus according to inventive concept 25, wherein the buffer chamber has a volume of at least 1 cc when a pressure of the gas in the buffer chamber is 25 cm H2O.

Inventive concept 27. The apparatus according to inventive concept 25, wherein a volume of the buffer chamber increases by at least 1 cc when a pressure of the gas in the buffer chamber increases from 25 cm H2O to 30 cm H2O.
Inventive concept 28. The apparatus according to inventive concept 27, wherein the volume of the buffer chamber increases by at least 2 cc when a pressure of the gas in the buffer chamber increases from 25 cm H2O to 30 cm H2O.
Inventive concept 29. The apparatus according to inventive concept 25, wherein the volume of the buffer chamber increases by no more than 5 cc when a pressure of the gas in the buffer chamber increases from 25 cm H2O to 30 cm H2O.
Inventive concept 30. The apparatus according to inventive concept 1, wherein the fluid reservoir contains some of the gas, and wherein the liquid column container is in fluid communication with the inflation lumen proximal port connector via the fluid reservoir.
Inventive concept 31. The apparatus according to inventive concept 30, wherein the fluid reservoir extends to the inflation lumen proximal port connector, and includes at least one wall that includes a volume-compensation movable wall, which is in pressure communication with the atmosphere.
Inventive concept 32. The apparatus according to inventive concept 31, wherein the volume-compensation movable wall include a flexible membrane.
Inventive concept 33. The apparatus according to inventive concept 31,

wherein the fluid reservoir includes a buffer chamber, which is shaped so as to define a chamber inlet port that is in fluid communication with the inflation lumen proximal port connector, and

wherein the buffer chamber includes the at least one wall that includes the volume-compensation movable wall.
Inventive concept 34. The apparatus according to inventive concept 33, wherein the buffer chamber has a volume of at least 2 cc when the gas in the buffer chamber is at a pressure of 25 cm H2O.
Inventive concept 35. The apparatus according to inventive concept 33, wherein a volume of the buffer chamber increases by at least 1 cc when a pressure of the gas in the buffer chamber increases from 25 cm H2O to 30 cm H2O.
Inventive concept 36. The apparatus according to inventive concept 33, wherein a volume of the buffer chamber increases by at least 1 cc when a pressure of the gas in the buffer chamber increases from 25 cm H2O to 28 cm H2O.
Inventive concept 37. The apparatus according to inventive concept 33, wherein a volume of the buffer chamber increases by at less than 3 cc when a pressure of the gas in the buffer chamber increases from 25 cm H2O to 28 cm H2O.
Inventive concept 38. The apparatus according to inventive concept 1, wherein the liquid column container includes a dissolvable wall portion that is dissolvable in water.
Inventive concept 39. The apparatus according to inventive concept 38, wherein the dissolvable wall portion defines a perforation therethrough that is configured to become permeable to the liquid through the perforation after total time of less than 30 days of contact with the liquid.
Inventive concept 40. The apparatus according to inventive concept 38,

wherein the cuff pressure stabilizer includes a gas container, which (a) extends to the inflation lumen proximal port connector, (b) contains some of the gas, and (c) is not in liquid communication with the fluid reservoir,

wherein the gas container includes a buffer chamber, which is shaped so as to define a chamber inlet port that is in fluid communication with the inflation lumen proximal port connector, and

wherein when the liquid column container is oriented in an aligned orientation in which the pressure indicia markings reflect, to within 1 cm H2O, pressure of the gas in the buffer chamber at least in a relevant pressure range of 23-27 cm H2O:
the dissolvable wall portion is disposed at least partially below an axial location along the liquid column container corresponding to a pressure of the gas in the buffer chamber of 23 cm H2O.
Inventive concept 41. The apparatus according to inventive concept 40, wherein when the liquid column container is oriented in the aligned orientation and the pressure of the gas in the buffer chamber equals ambient air pressure, the dissolvable wall portion is disposed above a liquid upper surface of the liquid in the liquid column container.
Inventive concept 42. The apparatus according to inventive concept 38,

wherein the fluid reservoir contains some of the gas, and

wherein when the liquid column container is oriented in an aligned orientation in which the pressure indicia markings reflect, to within 1 cm H2O, pressure of the gas in the fluid reservoir at least in a relevant pressure range of 23-27 cm H2O:
the dissolvable wall portion is disposed at least partially below an axial location along the liquid column container corresponding to a pressure of the gas in the fluid reservoir of 23 cm H2O.
Inventive concept 43. The apparatus according to inventive concept 42, wherein when the liquid column container is oriented in the aligned orientation and the pressure of the gas in the fluid reservoir equals ambient air pressure, the dissolvable wall portion is disposed above a liquid upper surface of the liquid in the liquid column container.
Inventive concept 44. The apparatus according to inventive concept 1, wherein the liquid column container (a) has a largest inner dimension equal to a greatest distance, in a plane perpendicular to a longitudinal axis of the liquid column container, between any two points within the liquid column container, and (b) can encompass a largest circle in the plane, and wherein at most or all of axial locations along the liquid column container, a ratio of (a) the largest inner dimension to (b) the diameter of the circle equals at least 2:1.
Inventive concept 45. The apparatus according to inventive concept 1, wherein the pressure indicia markings are distributed evenly throughout at least a relevant pressure range of 23-27 cm H2O.
Inventive concept 46. The apparatus according to inventive concept 1,

wherein the liquid column container has first and second ends at opposite ends of the liquid column container,

wherein the liquid column container is in fluid communication with the fluid reservoir via the first end, and

wherein the at least one site is at the second end, and the liquid column container is open to the atmosphere at the second end.
Inventive concept 47. The apparatus according to inventive concept 1, wherein the liquid column container has a length greater than 5 cm and less than 30 cm.
Inventive concept 48. The apparatus according to any one of inventive concepts 1-47, wherein the catheter is a tracheal ventilation tube, and wherein the apparatus is for use with the tracheal ventilation tube.
Inventive concept 49. The apparatus according to inventive concept 48, further including the tracheal ventilation tube, which includes the inflatable cuff, the inflation lumen, and the inflation lumen proximal port.
Inventive concept 50. The apparatus according to any one of inventive concepts 1-47, wherein the inflation lumen proximal port connector includes a male conical fitting with a taper.
Inventive concept 51. The apparatus according to inventive concept 50, wherein the taper is at least a 5% taper.
Inventive concept 52. The apparatus according to inventive concept 51, wherein the taper is a 6% taper, and the male conical fitting with the 6% taper complies with International Standard ISO 594-1:1986.

There is still further provided, in accordance with an inventive concept 53 of the present invention, apparatus for use in contact with the atmosphere of the Earth and for use with a gas and a catheter having an inflatable cuff, an inflation lumen, and an inflation lumen proximal port, the apparatus including a cuff pressure stabilizer, which includes:

an inflation lumen proximal port connector, which is shaped to form an air-tight seal with the inflation lumen proximal port of the catheter;

a gas inlet, which is in fluid communication with the inflation lumen proximal port connector;

a fluid reservoir, which has a volume of at least 2 cc, and which contains some of the gas;

a liquid column container, which is in fluid communication with the gas inlet via the fluid reservoir; and

a liquid, which is contained (a) in the fluid reservoir, (b) in the liquid column container, or (c) partially in the fluid reservoir and partially in the liquid column container,

wherein the cuff pressure stabilizer has a plurality of pressure indicia markings distributed along the liquid column container for measuring a height of the liquid in the liquid column container, and

wherein when the liquid column container is oriented in an aligned orientation in which the pressure indicia markings reflect, to within 1 cm H2O, pressure of the gas in the fluid reservoir at least in a relevant pressure range of 23-27 cm H2O:
the liquid column container has an inner cross-sectional area, measured in a horizontal plane, of at least 0.25 cm2 at a plurality of axial locations along the liquid column container corresponding to the pressure of the gas in the fluid reservoir at a respective plurality of pressures in the relevant pressure range.
Inventive concept 54. The apparatus according to inventive concept 53, wherein the inner cross-sectional area is at least 0.25 cm2 at most or all axial locations along the liquid column container corresponding to the pressure of the gas in the fluid reservoir at the respective plurality of pressures in the relevant pressure range, when the liquid column container is oriented in the aligned orientation.
Inventive concept 55. The apparatus according to inventive concept 53, wherein the inner cross-sectional area is at least 0.5 cm2 at the plurality of axial locations along the liquid column container corresponding to the pressure of the gas in the fluid reservoir at the respective plurality of pressures in the relevant pressure range, when the liquid column container is oriented in the aligned orientation.
Inventive concept 56. The apparatus according to inventive concept 55, wherein the inner cross-sectional area is at least 1 cm2 at the plurality of axial locations along the liquid column container corresponding to the pressure of the gas in the fluid reservoir at the respective plurality of pressures in the relevant pressure range, when the liquid column container is oriented in the aligned orientation.
Inventive concept 57. The apparatus according to inventive concept 53, wherein the pressure indicia markings are distributed evenly throughout at least the relevant pressure range.
Inventive concept 58. The apparatus according to inventive concept 53, wherein when the liquid column container is oriented in the aligned orientation, an average inner cross-sectional area at all axial locations along the liquid column container corresponding to the pressure of the gas in the fluid reservoir at the respective plurality of pressures in the relevant range is less than the inner cross-sectional area of the liquid column container, measured in the horizontal plane, at at least one axial location along the liquid column container corresponding to a pressure of the gas in the fluid reservoir of between 28 and 25 cm H2O.
Inventive concept 59. The apparatus according to inventive concept 53, wherein a distance between a highest point of the liquid column container and a lowest point of the fluid reservoir is between 10 and 20 cm, when the liquid column container is oriented in an aligned orientation.
Inventive concept 60. The apparatus according to inventive concept 53, wherein a central longitudinal axis of the liquid column container is perpendicular to the horizontal plane, when the liquid column container is oriented in the aligned orientation.
Inventive concept 61. The apparatus according to inventive concept 53, wherein a central longitudinal axis of the liquid column container is not perpendicular to the horizontal plane, when the liquid column container is oriented in the aligned orientation.
Inventive concept 62. The apparatus according to inventive concept 53, wherein the volume of the fluid reservoir is at least 3 cc.
Inventive concept 63. The apparatus according to inventive concept 62, wherein the volume of the fluid reservoir is at least 4 cc.
Inventive concept 64. The apparatus according to inventive concept 53, wherein the volume of the fluid reservoir is less than 5 cc.
Inventive concept 65. The apparatus according to inventive concept 53, wherein the cuff pressure stabilizer further includes a container-sealing element that is removably disposed between the fluid reservoir and the liquid column container so as to prevent fluid communication between the fluid reservoir and the liquid container column.
Inventive concept 66. The apparatus according to any one of inventive concepts 105-117, wherein when the liquid column container is oriented in the aligned orientation, an average inner cross-sectional area, measured in the horizontal plane, at all axial locations along the liquid column container corresponding to the pressure of the gas in the fluid reservoir at the respective plurality of pressures in the relevant pressure range is greater than the product of (a) a factor greater than one and (b) an average inner cross-sectional area of the liquid column container, measured in the horizontal plane, at all axial locations along the liquid column container corresponding to pressure of the gas in the fluid reservoir of less than 20 cm H2O.
Inventive concept 67. The apparatus according to inventive concept 66, wherein the factor is 200%.
Inventive concept 68. The apparatus according to inventive concept 67, wherein the factor is 500%.
Inventive concept 69. The apparatus according to inventive concept 66, wherein when the liquid column container is oriented in the aligned orientation, the inner cross-sectional area at the plurality of axial locations along the liquid column container corresponding to the pressure of the gas in the fluid reservoir at the respective plurality of pressures in the relevant pressure range equals at least 200% of an average inner cross-sectional area of the liquid column container, measured in the horizontal plane, at most or all axial locations along the liquid column container corresponding to a pressure of the gas in the fluid reservoir of between 5 and 15 cm H2O.
Inventive concept 70. The apparatus according to inventive concept 69, wherein the average inner cross-sectional area is less than 0.09 cm2 at most or all axial locations along the liquid column container corresponding to a pressure of the gas in the fluid reservoir of between 5 and 15 cm H2O, when the liquid column container is oriented in the aligned orientation.
Inventive concept 71. The apparatus according to any one of inventive concepts 105-117, wherein the inflation lumen proximal port connector includes a male conical fitting with a taper.
Inventive concept 72. The apparatus according to inventive concept 71, wherein the taper is at least a 5% taper.
Inventive concept 73. The apparatus according to inventive concept 72, wherein the taper is a 6% taper, and the male conical fitting with the 6% taper complies with International Standard ISO 594-1:1986.
Inventive concept 74. The apparatus according to any one of inventive concepts 105-117, wherein the liquid has a density of between 0.8 and 12 g/cm3 at 4 degrees Celsius at 1 atm.
Inventive concept 75. The apparatus according to inventive concept 74, wherein the density is between 1.5 and 5 g/cm3.
Inventive concept 76. The apparatus according to inventive concept 75, wherein the density is between 2 and 4 g/cm3.
Inventive concept 77. The apparatus according to inventive concept 74, wherein the density is between 2.5 and 3.5 g/cm3.
Inventive concept 78. The apparatus according to inventive concept 74, wherein the liquid is selected from the group consisting of: sodium polytungstate, sodium metatungstate, lithium polytungstate, and lithium metatungstate.
Inventive concept 79. The apparatus according to any one of inventive concepts 105-117, wherein the liquid column container is open to the atmosphere at at least one site along the liquid column container.
Inventive concept 80. The apparatus according to inventive concept 79,

wherein the liquid column container has first and second ends at opposite ends of the liquid column container,

wherein the liquid column container is in fluid communication with the fluid reservoir via the first end, and

wherein the at least one site is at the second end, and the liquid column container is open to the atmosphere at the second end.
Inventive concept 81. The apparatus according to inventive concept 79, wherein the liquid column container defines an opening having an area of between 0.09 and 1 mm2, and wherein the liquid column container is open to the atmosphere via the opening.
Inventive concept 82. The apparatus according to inventive concept 81, wherein the cuff pressure stabilizer further includes a sealing element that is removably disposed so as to seal the opening.
Inventive concept 83. The apparatus according to any one of inventive concepts 105-117, wherein the cuff pressure stabilizer is arranged such that if the inflatable cuff is squeezed to a high threshold pressure, further squeezing of the inflatable cuff releases gas bubbles through the liquid in the liquid column container and prevents the pressure from further increasing within the inflatable cuff.
Inventive concept 84. The apparatus according to any one of inventive concepts 105-117, wherein the cuff pressure stabilizer further includes an orientation-sensitive valve assembly, which includes a valve, and which is arranged to automatically assume:

an open state when an orientation of the cuff pressure stabilizer differs from the aligned orientation by no more than a constant number of degrees, the constant equal to between 5 and 45 degrees, and

a reduced-flow state when the orientation of the cuff pressure stabilizer differs from the aligned orientation by more than the constant number of degrees,

wherein the valve is configured to reduce fluid communication thereacross by at least 90% when in the reduced-flow state compared to when in the open state.
Inventive concept 85. The apparatus according to inventive concept 84, wherein the valve is arranged in a fluid path between the inflation lumen proximal port connector and the fluid reservoir.
Inventive concept 86. The apparatus according to inventive concept 84, wherein the orientation-sensitive valve assembly is mechanical and non-electrical.
Inventive concept 87. The apparatus according to inventive concept 84, wherein the orientation-sensitive valve assembly includes an orientation sensor, which is configured to sense the orientation of the cuff pressure stabilizer.
Inventive concept 88. The apparatus according to inventive concept 84, wherein the valve is configured to entirely block fluid communication thereacross when in the reduced-flow state.
Inventive concept 89. The apparatus according to any one of inventive concepts 105-117, wherein the catheter is a tracheal ventilation tube, and wherein the apparatus is for use with the tracheal ventilation tube.
Inventive concept 90. The apparatus according to inventive concept 89, further including the tracheal ventilation tube, which includes the inflatable cuff, the inflation lumen, and the inflation lumen proximal port.
Inventive concept 91. The apparatus according to inventive concept 90,

wherein the tracheal ventilation tube further includes an inflating tube, which couples the inflation lumen in fluid communication with the inflation lumen proximal port,

wherein the cuff pressure stabilizer further includes:
an inflation inlet port;
a first connector tube, which couples the inflation lumen proximal port connector in fluid communication with the inflation inlet port; and
a second connector tube, which couples the gas inlet in fluid communication with the inflation inlet port, such that the inflation lumen proximal port connector is in fluid communication with the gas inlet via the first connector tube and the second connector tube, and

wherein when (a) the inflation lumen proximal port connector forms the air-tight seal with the inflation lumen proximal port of the tracheal ventilation tube and (b) the pressure of the gas in the fluid reservoir is 10 cm H2O, (i) a first combined air-flow resistance between the inflation inlet port and an interior of the inflatable cuff equals between 80% and 120% of (ii) a second combined air-flow resistance between inflation inlet port and the fluid reservoir.
Inventive concept 92. The apparatus according to inventive concept 89,

wherein the tracheal ventilation tube further includes an inflating tube, which couples the inflation lumen in fluid communication with the inflation lumen proximal port,

wherein the cuff pressure stabilizer further includes one or more connector tubes, which couple the inflation lumen proximal port connector in fluid communication with the gas inlet, and

wherein when the inflation lumen proximal port connector forms the air-tight seal with the inflation lumen proximal port of the tracheal ventilation tube, a combined air-flow resistance between an interior of the inflatable cuff and the fluid reservoir is such that a transient pressure difference of 5 cm H2O between the interior of the inflatable cuff and the fluid reservoir results in less than 0.01 cc per second fluid flow from the inflatable cuff into the fluid reservoir.
Inventive concept 93. The apparatus according to inventive concept 92, wherein the cuff pressure stabilizer further includes a switch that sets flow states of the cuff pressure stabilizer, including:

a flow-limiting state, in which the combined air-flow resistance between the interior of the inflatable cuff and the fluid reservoir is such that the transient pressure difference of 5 cm H2O between the interior of the inflatable cuff and the fluid reservoir results in less than 0.01 cc per second fluid flow from the inflatable cuff into the fluid reservoir, and

a fast-flow state, in which the combined air-flow resistance between the interior of the inflatable cuff and the fluid reservoir is such that the transient pressure difference of 5 cm H2O between the interior of the inflatable cuff and the fluid reservoir results in more than 0.02 cc per second fluid flow from the inflatable cuff into the fluid reservoir.
Inventive concept 94. The apparatus according to any one of inventive concepts 105-117, wherein the cuff pressure stabilizer does not include any membranes in contact with the liquid, and does not include any membranes in a fluid path between the liquid and the atmosphere.
Inventive concept 95. The apparatus according to any one of inventive concepts 105-117, wherein the cuff pressure stabilizer does not include a spring for measuring the pressure of the gas in the fluid reservoir.
Inventive concept 96. The apparatus according to any one of inventive concepts 105-117, wherein the cuff pressure stabilizer is for use with an IV pole, and wherein the cuff pressure stabilizer includes a coupling element selected from the group consisting of:

a hook or a loop, which is configured to automatically orient the liquid column container in the aligned orientation when hung from a hook of the IV pole, and

a squeezing coupler that is coupleable to a vertical or horizontal portion of the IV pole.

There is additionally provided, in accordance with an inventive concept 97 of the present invention, apparatus for use on the Earth with a gas and a catheter having an inflatable cuff, an inflation lumen, and an inflation lumen proximal port, the apparatus including a cuff pressure stabilizer, which includes:

an inflation lumen proximal port connector, which is shaped to form an air-tight seal with the inflation lumen proximal port of the catheter;

a gas inlet, which is in fluid communication with the inflation lumen proximal port connector;

a fluid reservoir, which has a volume of at least 2 cc, and which contains some of the gas;

a liquid column container, which is in fluid communication with the gas inlet via the fluid reservoir; and

a liquid, which is contained (a) in the fluid reservoir, (b) in the liquid column container, or (c) partially in the fluid reservoir and partially in the liquid column container,

wherein the cuff pressure stabilizer has a plurality of pressure indicia markings distributed along the liquid column container for measuring a height of the liquid in the liquid column container, and

wherein when the liquid column container is oriented in an aligned orientation in which the pressure indicia markings reflect, to within 1 cm H2O, pressure of the gas in the fluid reservoir at least in a relevant pressure range of 23-27 cm H2O, an inner cross-sectional area of the liquid column container, measured in a horizontal plane, at a plurality of axial locations along the liquid column container corresponding to the pressure of the gas in the fluid reservoir at a respective plurality of pressures in the relevant pressure range is greater than the product of (a) a factor equal to 200% and (b) an average inner cross-sectional area of the liquid column container, measured in the horizontal plane, at all axial locations along the liquid column container corresponding to a pressure of the gas in the fluid reservoir of between 5 and 15 cm H2O.
Inventive concept 98. The apparatus according to inventive concept 97, wherein the inner cross-sectional area of the liquid column container, at the plurality of axial locations along the liquid column container corresponding to the pressure of the gas in the fluid reservoir at the respective plurality of pressures in the relevant pressure range, is greater than the product of (a) a factor equal to 500% and (b) the average inner cross-sectional area of the liquid column container at all axial locations along the liquid column container corresponding to the pressure of the gas in the fluid reservoir of between 5 and 15 cm H2O.
Inventive concept 99. The apparatus according to inventive concept 97, wherein the pressure indicia markings are distributed evenly throughout at least the relevant pressure range.
Inventive concept 100. The apparatus according to inventive concept 97, wherein the average inner cross-sectional area is less than 0.09 cm2 at most or all axial locations along the liquid column container corresponding to a pressure of the gas in the fluid reservoir of between 5 and 15 cm H2O, when the liquid column container is oriented in the aligned orientation.
Inventive concept 101. The apparatus according to inventive concept 97, wherein when the liquid column container is oriented in the aligned orientation, the inner cross-sectional area at the axial location along the liquid column container corresponding to a pressure of the gas in the fluid reservoir of 25 cm H2O is less than the inner cross-sectional area of the liquid column container, measured in the horizontal plane, at at least one axial location along the liquid column container corresponding to a pressure of the gas in the fluid reservoir of between 28 and 35 cm H2O.
Inventive concept 102. The apparatus according to inventive concept 97, wherein the liquid has a density of between 1.5 and 12 g/cm3 at 4 degrees Celsius at 1 atm.
Inventive concept 103. The apparatus according to inventive concept 97, wherein the volume of the fluid reservoir is at least 3 cc.
Inventive concept 104. The apparatus according to inventive concept 103, wherein the volume of the fluid reservoir is at least 4 cc.
Inventive concept 105. The apparatus according to inventive concept 97, wherein the volume of the fluid reservoir is less than 5 cc.
Inventive concept 106. The apparatus according to inventive concept 97, wherein a central longitudinal axis of the liquid column container is perpendicular to the horizontal plane, when the liquid column container is oriented in the aligned orientation.
Inventive concept 107. The apparatus according to inventive concept 97, wherein a central longitudinal axis of the liquid column container is not perpendicular to the horizontal plane, when the liquid column container is oriented in the aligned orientation.
Inventive concept 108. The apparatus according to any one of inventive concepts 154-165,

wherein the liquid column container is shaped so as to define a wider portion and a narrower portion axially between the wider portion and the fluid reservoir,

wherein at each of the axial locations along the narrower portion of the liquid column container, the liquid column container (a) has a largest inner dimension equal to a greatest distance in the horizontal plane between any two points within the liquid column container, and (b) can encompass a largest circle in the horizontal plane, and

wherein at most or all of the axial locations along the narrower portion of the liquid column container, a ratio of (a) the largest inner dimension to (b) the diameter of the circle equals at least 2:1.
Inventive concept 109. The apparatus according to inventive concept 108, wherein the ratio equals at least 2:1 at all the axial locations along the narrower portion of the liquid column container.
Inventive concept 110. The apparatus according to inventive concept 108, wherein the ratio equals at least 4:1 at most or all of the axial locations along the narrower portion of the liquid column container.
Inventive concept 111. The apparatus according to inventive concept 110, wherein the ratio equals at least 8:1 at most or all of the axial locations along the narrower portion of the liquid column container.
Inventive concept 112. The apparatus according to inventive concept 108, wherein the largest inner dimension equals at least 4 mm at most or all of the axial locations along the narrower portion of the liquid column container.
Inventive concept 113. The apparatus according to inventive concept 112, wherein the largest inner dimension equals at least 6 mm at most or all of the axial locations along the narrower portion of the liquid column container.
Inventive concept 114. The apparatus according to inventive concept 113, wherein the largest inner dimension equals at least 8 mm at most or all of the axial locations along the narrower portion of the liquid column container.
Inventive concept 115. The apparatus according to inventive concept 108, wherein the diameter of the circle is no more than 4 mm at most or all of the axial locations along the narrower portion of the liquid column container.
Inventive concept 116. The apparatus according to inventive concept 115, wherein the diameter of the circle is no more than 2 mm at most or all of the axial locations along the narrower portion of the liquid column container.
Inventive concept 117. The apparatus according to inventive concept 116, wherein the diameter of the circle is no more than 1 mm at most or all of the axial locations along the narrower portion of the liquid column container.
Inventive concept 118. The apparatus according to inventive concept 117, wherein the diameter of the circle is no more than 0.5 mm at most or all of the axial locations along the narrower portion of the liquid column container.
Inventive concept 119. The apparatus according to inventive concept 108, wherein, at most or all of the axial locations along the narrower portion of the liquid column container, the liquid column container has a non-circular cross-sectional shape.
Inventive concept 120. The apparatus according to inventive concept 119, wherein, at most or all of the axial locations along the narrower portion of the liquid column container, the cross-sectional shape of the liquid column container is selected from the group of shapes consisting of: a rectangle, an oblong shape, an ellipse, and a crescent.
Inventive concept 121. The apparatus according to any one of inventive concepts 154-165,

wherein the liquid column container is shaped so as to define a wider portion and a narrower portion axially between the wider portion and the fluid reservoir, and

wherein at each of the axial locations along the narrower portion of the liquid column container, at least 80% of the inner cross-sectional area is within 1 mm of an inner surface of the liquid column container.
Inventive concept 122. The apparatus according to inventive concept 121, wherein at each of the axial locations along the narrower portion of the liquid column container, at least 80% of the inner cross-sectional area is within 0.5 mm of the inner surface of the liquid column container.
Inventive concept 123. The apparatus according to inventive concept 121, wherein at each of the axial locations along the narrower portion of the liquid column container, less than 10% of the inner cross-sectional area is within 0.1 mm of the inner surface of the liquid column container.
Inventive concept 124. The apparatus according to inventive concept 121, wherein the inner cross-sectional area is less than 0.09 cm2 at most or all axial locations along the narrower portion of the liquid column container, when the liquid column container is oriented in the aligned orientation.
Inventive concept 125. The apparatus according to inventive concept 121, wherein, at most or all of the axial locations along the narrower portion of the liquid column container, the liquid column container has a non-circular cross-sectional shape.
Inventive concept 126. The apparatus according to inventive concept 125, wherein, at most or all of the axial locations along the narrower portion of the liquid column container, the cross-sectional shape of the liquid column container is selected from the group of shapes consisting of: a rectangle, an oblong shape, an ellipse, and a crescent.
Inventive concept 127. The apparatus according to any one of inventive concepts 154-165,

wherein the liquid column container is shaped so as to define a wider portion and a narrower portion axially between the wider portion and the fluid reservoir, and

wherein the cuff pressure stabilizer is arranged to provide an adjustable distance between the wider portion of the liquid column container and the fluid reservoir.
Inventive concept 128. The apparatus according to inventive concept 127, wherein the cuff pressure stabilizer is arranged such that the adjustable distance can vary by at least 1 cm.
Inventive concept 129. The apparatus according to inventive concept 127, wherein the cuff pressure stabilizer is arranged such that the adjustable distance can vary by at least the quotient of (a) 3 cm divided by (b) the specific gravity of the liquid with reference to water at 4 degrees Celsius at 1 atm.
Inventive concept 130. The apparatus according to inventive concept 127, wherein an average cross-sectional area of the wider portion, measured in the horizontal plane, equals at least 200% of an average cross-sectional area of the narrower portion, measured in the horizontal plane.
Inventive concept 131. The apparatus according to inventive concept 127, wherein the cuff pressure stabilizer includes a mechanical user control element, which is arranged to set the adjustable distance.
Inventive concept 132. The apparatus according to inventive concept 127, wherein the cuff pressure stabilizer includes a casing, and wherein the wider portion of the liquid column container is axially-slidably coupled to the casing.
Inventive concept 133. The apparatus according to inventive concept 132, wherein the cuff pressure stabilizer includes a mechanical user control element, which is arranged to set an axial position of the wider portion of the liquid column container with respect to the casing.
Inventive concept 134. The apparatus according to inventive concept 132, wherein the casing has the pressure indicia markings.
Inventive concept 135. The apparatus according to inventive concept 134, wherein the wider portion of the liquid column container has a target-pressure indicator marker, which is axially slidable with respect to the pressure indicia markings.
Inventive concept 136. The apparatus according to inventive concept 134, wherein the cuff pressure stabilizer includes a mechanical user control element, which is arranged to set an axial position of the wider portion of the liquid column container with respect to the casing.
Inventive concept 137. The apparatus according to inventive concept 127, wherein at least an axial portion of the narrower portion of the liquid column container is flexible, so as to provide a variable axial length to the narrower portion.
Inventive concept 138. The apparatus according to inventive concept 137, wherein the at least an axial portion of the narrower portion of the liquid column container is elastic.
Inventive concept 139. The apparatus according to inventive concept 127, wherein at least an axial portion of the narrower portion of the liquid column container is telescopically adjustable, so as to provide a variable axial length to the narrower portion.
Inventive concept 140. The apparatus according to inventive concept 127, wherein the cuff pressure stabilizer includes a casing, and wherein the fluid reservoir is axially-slidably coupled to the casing.
Inventive concept 141. The apparatus according to any one of inventive concepts 154-165, wherein the catheter is a tracheal ventilation tube, and wherein the apparatus is for use with the tracheal ventilation tube.
Inventive concept 142. The apparatus according to inventive concept 141, further including the tracheal ventilation tube, which includes the inflatable cuff, the inflation lumen, and the inflation lumen proximal port.
Inventive concept 143. The apparatus according to any one of inventive concepts 154-165, wherein the inflation lumen proximal port connector includes a male conical fitting with a taper.
Inventive concept 144. The apparatus according to inventive concept 143, wherein the taper is at least a 5% taper.
Inventive concept 145. The apparatus according to inventive concept 144, wherein the taper is a 6% taper, and the male conical fitting with the 6% taper complies with International Standard ISO 594-1:1986.

There is yet additionally provided, in accordance with an inventive concept 146 of the present invention, apparatus for use on the Earth with a gas, an inflatable chamber, an inflation lumen, and an inflation lumen proximal port in fluid communication with the inflatable chamber via the inflation lumen, the apparatus including a chamber pressure stabilizer, which includes:

an inflation lumen proximal port connector, which is shaped to form an air-tight seal with the inflation lumen proximal port;

a gas inlet, which is in fluid communication with the inflation lumen proximal port connector;

a fluid reservoir, which has a volume of at least 2 cc, and which contains some of the gas;

a liquid column container, which is in fluid communication with the gas inlet via the fluid reservoir, and which is shaped so as to define a wider portion and a narrower portion axially between the wider portion and the fluid reservoir, wherein the chamber pressure stabilizer is arranged to provide an adjustable distance between the wider portion of the liquid column container and the fluid reservoir; and

a liquid, which is contained (a) in the fluid reservoir, (b) in the liquid column container, or (c) partially in the fluid reservoir and partially in the liquid column container.
Inventive concept 147. The apparatus according to inventive concept 146, wherein the chamber pressure stabilizer is arranged such that the adjustable distance can vary by at least 1 cm.
Inventive concept 148. The apparatus according to inventive concept 146, wherein the chamber pressure stabilizer is arranged such that the adjustable distance can vary by at least the quotient of (a) 3 cm divided by (b) the specific gravity of the liquid with reference to water at 4 degrees Celsius at 1 atm.
Inventive concept 149. The apparatus according to inventive concept 146, wherein an average cross-sectional area of the wider portion, measured in a plane perpendicular to a longitudinal axis of the liquid column container, equals at least 200% of an average cross-sectional area of the narrower portion, measured in the plane.
Inventive concept 150. The apparatus according to inventive concept 149, wherein the average cross-sectional area of the wider portion equals at least 300% of the average cross-sectional area of the narrower portion.
Inventive concept 151. The apparatus according to inventive concept 146, wherein the chamber pressure stabilizer includes a mechanical user control element, which is arranged to set the adjustable distance.
Inventive concept 152. The apparatus according to inventive concept 146, wherein the chamber pressure stabilizer includes a casing, and wherein the wider portion of the liquid column container is axially-slidably coupled to the casing.
Inventive concept 153. The apparatus according to inventive concept 152, wherein the chamber pressure stabilizer includes a mechanical user control element, which is arranged to set an axial position of the wider portion of the liquid column container with respect to the casing.
Inventive concept 154. The apparatus according to inventive concept 152, wherein the casing has a plurality of pressure indicia markings distributed along the liquid column container for measuring a height of the liquid in the liquid column container.
Inventive concept 155. The apparatus according to inventive concept 154, wherein the chamber pressure stabilizer includes a mechanical user control element, which is arranged to set an axial position of the wider portion of the liquid column container with respect to the casing.
Inventive concept 156. The apparatus according to inventive concept 154, wherein the wider portion of the liquid column container has a target-pressure indicator marker, which is axially slidable with respect to the pressure indicia markings.
Inventive concept 157. The apparatus according to inventive concept 152, wherein the chamber pressure stabilizer includes a mechanical user control element, which is arranged to set an axial position of the wider portion of the liquid column container with respect to the casing.
Inventive concept 158. The apparatus according to inventive concept 146, wherein at least an axial portion of the narrower portion of the liquid column container is flexible, so as to provide a variable axial length to the narrower portion.
Inventive concept 159. The apparatus according to inventive concept 158, wherein the at least an axial portion of the narrower portion of the liquid column container is elastic.
Inventive concept 160. The apparatus according to inventive concept 146, wherein at least an axial portion of the narrower portion of the liquid column container is telescopically adjustable, so as to provide a variable axial length to the narrower portion.
Inventive concept 161. The apparatus according to inventive concept 146, wherein the chamber pressure stabilizer includes a casing, and wherein the fluid reservoir is axially-slidably coupled to the casing.
Inventive concept 162. The apparatus according to inventive concept 146, wherein the volume of the fluid reservoir is at least 3 cc.
Inventive concept 163. The apparatus according to inventive concept 162, wherein the volume of the fluid reservoir is at least 4 cc.
Inventive concept 164. The apparatus according to inventive concept 146, wherein the volume of the fluid reservoir is less than 5 cc.
Inventive concept 165. The apparatus according to inventive concept 146, wherein the liquid has a density of between 1.5 and 12 g/cm3 at 4 degrees Celsius at 1 atm.
Inventive concept 166. The apparatus according to any one of inventive concepts 205-226, wherein the chamber pressure stabilizer has a plurality of pressure indicia markings distributed along the liquid column container for measuring a height of the liquid in the liquid column container.
Inventive concept 167. The apparatus according to any one of inventive concepts 205-226,

wherein at each of all axial locations along the narrower portion of the liquid column container, the liquid column container (a) has a largest inner dimension equal to a greatest distance, in a plane perpendicular to a longitudinal axis of the liquid column container, between any two points within the liquid column container, and (b) the liquid column container can encompass a largest circle in the plane, and

wherein at most or all of the axial locations along the narrower portion of the liquid column container, a ratio of (a) the largest inner dimension to (b) the diameter of the circle equals at least 2:1.
Inventive concept 168. The apparatus according to inventive concept 167, wherein the ratio equals at least 2:1 at all the axial locations along the narrower portion of the liquid column container.
Inventive concept 169. The apparatus according to inventive concept 167, wherein the ratio equals at least 4:1 at most or all of the axial locations along the narrower portion of the liquid column container.
Inventive concept 170. The apparatus according to inventive concept 169, wherein the ratio equals at least 8:1 at most or all of the axial locations along the narrower portion of the liquid column container.
Inventive concept 171. The apparatus according to inventive concept 167, wherein the largest inner dimension equals at least 4 mm at most or all of the axial locations along the narrower portion of the liquid column container.
Inventive concept 172. The apparatus according to inventive concept 171, wherein the largest inner dimension equals at least 6 mm at most or all of the axial locations along the narrower portion of the liquid column container.
Inventive concept 173. The apparatus according to inventive concept 172, wherein the largest inner dimension equals at least 8 mm at most or all of the axial locations along the narrower portion of the liquid column container.
Inventive concept 174. The apparatus according to inventive concept 167, wherein the diameter of the circle is no more than 4 mm at most or all of the axial locations along the narrower portion of the liquid column container.
Inventive concept 175. The apparatus according to inventive concept 174, wherein the diameter of the circle is no more than 2 mm at most or all of the axial locations along the narrower portion of the liquid column container.
Inventive concept 176. The apparatus according to inventive concept 175, wherein the diameter of the circle is no more than 1 mm at most or all of the axial locations along the narrower portion of the liquid column container.
Inventive concept 177. The apparatus according to inventive concept 176, wherein the diameter of the circle is no more than 0.5 mm at most or all of the axial locations along the narrower portion of the liquid column container.
Inventive concept 178. The apparatus according to inventive concept 167, wherein, at most or all of the axial locations along the narrower portion of the liquid column container, the liquid column container has a non-circular cross-sectional shape.
Inventive concept 179. The apparatus according to inventive concept 178, wherein, at most or all of the axial locations along the narrower portion of the liquid column container, the cross-sectional shape of the liquid column container is selected from the group of shapes consisting of: a rectangle, an oblong shape, an ellipse, and a crescent.
Inventive concept 180. The apparatus according to any one of inventive concepts 205-226, wherein at each of all axial locations along the narrower portion of the liquid column container, at least 80% of an inner cross-sectional area, in a plane perpendicular to a longitudinal axis of the liquid column container, is within 1 mm of an inner surface of the liquid column container.
Inventive concept 181. The apparatus according to inventive concept 180, wherein at each of the axial locations along the narrower portion of the liquid column container, at least 80% of the inner cross-sectional area is within 0.5 mm of the inner surface of the liquid column container.
Inventive concept 182. The apparatus according to inventive concept 180, wherein at each of the axial locations along the narrower portion of the liquid column container, less than 10% of the inner cross-sectional area is within 0.1 mm of the inner surface of the liquid column container.
Inventive concept 183. The apparatus according to inventive concept 180, wherein an inner cross-sectional area is less than 0.09 cm2 at most or all axial locations along the narrower portion of the liquid column container.
Inventive concept 184. The apparatus according to inventive concept 180, wherein, at most or all of the axial locations along the narrower portion of the liquid column container, the liquid column container has a non-circular cross-sectional shape.
Inventive concept 185. The apparatus according to inventive concept 184, wherein, at most or all of the axial locations along the narrower portion of the liquid column container, the cross-sectional shape of the liquid column container is selected from the group of shapes consisting of: a rectangle, an oblong shape, an ellipse, and a crescent.
Inventive concept 186. The apparatus according to any one of inventive concepts 205-226, wherein the liquid column container is open to the atmosphere at at least one site along the liquid column container.
Inventive concept 187. The apparatus according to inventive concept 186,

wherein the liquid column container has first and second ends at opposite ends of the liquid column container,

wherein the liquid column container is in fluid communication with the fluid reservoir via the first end, and

wherein the at least one site is at the second end, and the liquid column container is open to the atmosphere at the second end.
Inventive concept 188. The apparatus according to inventive concept 186, wherein the liquid column container defines an opening having an area of between 0.09 and 1 mm2, and wherein the liquid column container is open to the atmosphere via the opening.
Inventive concept 189. The apparatus according to inventive concept 188, wherein the chamber pressure stabilizer further includes a sealing element that is removably disposed so as to seal the opening.
Inventive concept 190. The apparatus according to any one of inventive concepts 205-226, wherein the chamber pressure stabilizer is arranged such that if the inflatable chamber is squeezed to a high threshold pressure, further squeezing of the inflatable chamber releases gas bubbles through the liquid in the liquid column container prevents the pressure from further increasing within the inflatable chamber.
Inventive concept 191. The apparatus according to any one of inventive concepts 205-226, wherein the inflatable chamber is an inflatable cuff, wherein the apparatus is for use with a tracheal ventilation tube having the inflatable cuff, the inflation lumen, and the inflation lumen proximal port, and wherein the inflation lumen proximal port connector is shaped to form the air-tight seal with the inflation lumen proximal port of the tracheal ventilation tube.
Inventive concept 192. The apparatus according to inventive concept 191, further including the tracheal ventilation tube, which includes the inflatable cuff, the inflation lumen, and the inflation lumen proximal port.
Inventive concept 193. The apparatus according to inventive concept 191, wherein the inflation lumen proximal port connector includes a male conical fitting with a taper.
Inventive concept 194. The apparatus according to inventive concept 193, wherein the taper is at least a 5% taper.
Inventive concept 195. The apparatus according to inventive concept 194, wherein the taper is a 6% taper, and the male conical fitting with the 6% taper complies with International Standard ISO 594-1:1986.

There is also provided, in accordance with an inventive concept 196 of the present invention, apparatus for use on the Earth with a gas, an inflatable chamber, an inflation lumen, and an inflation lumen proximal port in fluid communication with the inflatable chamber via the inflation lumen, the apparatus including a chamber pressure stabilizer, which includes:

an inflation lumen proximal port connector, which is shaped to form an air-tight seal with the inflation lumen proximal port;

a gas inlet, which is in fluid communication with the inflation lumen proximal port connector;

a fluid reservoir, which has a volume of at least 2 cc, and which contains some of the gas;

a liquid column container, which is in fluid communication with the gas inlet via the fluid reservoir, and which is shaped so as to define a wider portion and a narrower portion axially between the wider portion and the fluid reservoir; and

a liquid, which is contained (a) in the fluid reservoir, (b) in the liquid column container, or (c) partially in the fluid reservoir and partially in the liquid column container,

wherein at each of all axial locations along the narrower portion of the liquid column container, the liquid column container (a) has a largest inner dimension equal to a greatest distance, in a plane perpendicular to a longitudinal axis of the liquid column container, between any two points within the liquid column container, and (b) can encompass a largest circle in the plane, and

wherein at most or all of the axial locations along the narrower portion of the liquid column container, a ratio of (a) the largest inner dimension to (b) the diameter of the circle equals at least 2:1.
Inventive concept 197. The apparatus according to inventive concept 196, wherein an average cross-sectional area of the wider portion, measured in the plane, equals at least 200% of an average cross-sectional area of the narrower portion, measured in the plane.
Inventive concept 198. The apparatus according to inventive concept 197, wherein the average cross-sectional area of the wider portion equals at least 300% of the average cross-sectional area of the narrower portion.
Inventive concept 199. The apparatus according to inventive concept 196, wherein the ratio equals at least 2:1 at all the axial locations along the narrower portion of the liquid column container.
Inventive concept 200. The apparatus according to inventive concept 196, wherein the ratio equals at least 4:1 at most or all of the axial locations along the narrower portion of the liquid column container.
Inventive concept 201. The apparatus according to inventive concept 200, wherein the ratio equals at least 8:1 at most or all of the axial locations along the narrower portion of the liquid column container.
Inventive concept 202. The apparatus according to inventive concept 196, wherein the largest inner dimension equals at least 4 mm at most or all of the axial locations along the narrower portion of the liquid column container.
Inventive concept 203. The apparatus according to inventive concept 202, wherein the largest inner dimension equals at least 6 mm at most or all of the axial locations along the narrower portion of the liquid column container.
Inventive concept 204. The apparatus according to inventive concept 203, wherein the largest inner dimension equals at least 8 mm at most or all of the axial locations along the narrower portion of the liquid column container.
Inventive concept 205. The apparatus according to inventive concept 196, wherein the diameter of the circle is no more than 4 mm at most or all of the axial locations along the narrower portion of the liquid column container.
Inventive concept 206. The apparatus according to inventive concept 205, wherein the diameter of the circle is no more than 2 mm at most or all of the axial locations along the narrower portion of the liquid column container.
Inventive concept 207. The apparatus according to inventive concept 206, wherein the diameter of the circle is no more than 1 mm at most or all of the axial locations along the narrower portion of the liquid column container.
Inventive concept 208. The apparatus according to inventive concept 207, wherein the diameter of the circle is no more than 0.5 mm at most or all of the axial locations along the narrower portion of the liquid column container.
Inventive concept 209. The apparatus according to inventive concept 196, wherein, at most or all of the axial locations along the narrower portion of the liquid column container, the liquid column container has a non-circular cross-sectional shape.
Inventive concept 210. The apparatus according to inventive concept 209, wherein, at most or all of the axial locations along the narrower portion of the liquid column container, the cross-sectional shape of the liquid column container is selected from the group of shapes consisting of: a rectangle, an oblong shape, an ellipse, and a crescent.
Inventive concept 211. The apparatus according to any one of inventive concepts 257-271, wherein at each of the axial locations along the narrower portion of the liquid column container, at least 80% of an inner cross-sectional area, in a plane perpendicular to a longitudinal axis of the liquid column container, is within 1 mm of an inner surface of the liquid column container.
Inventive concept 212. The apparatus according to inventive concept 211, wherein at each of the axial locations along the narrower portion of the liquid column container, less than 10% of the inner cross-sectional area is within 0.1 mm of the inner surface of the liquid column container.
Inventive concept 213. The apparatus according to inventive concept 211, wherein an inner cross-sectional area is less than 0.09 cm2 at most or all axial locations along the narrower portion of the liquid column container.
Inventive concept 214. The apparatus according to inventive concept 211, wherein, at most or all of the axial locations along the narrower portion of the liquid column container, the liquid column container has a non-circular cross-sectional shape.
Inventive concept 215. The apparatus according to inventive concept 214, wherein, at most or all of the axial locations along the narrower portion of the liquid column container, the cross-sectional shape of the liquid column container is selected from the group of shapes consisting of: a rectangle, an oblong shape, an ellipse, and a crescent.
Inventive concept 216. The apparatus according to inventive concept 196, wherein the chamber pressure stabilizer is arranged to provide an adjustable distance between the wider portion of the liquid column container and the fluid reservoir.
Inventive concept 217. The apparatus according to inventive concept 216, wherein the chamber pressure stabilizer includes a casing, and wherein the wider portion of the liquid column container is axially-slidably coupled to the casing.
Inventive concept 218. The apparatus according to inventive concept 217, wherein the casing has a plurality of pressure indicia markings distributed along the liquid column container for measuring a height of the liquid in the liquid column container.
Inventive concept 219. The apparatus according to inventive concept 218, wherein the wider portion of the liquid column container has a target-pressure indicator marker, which is axially slidable with respect to the pressure indicia markings.
Inventive concept 220. The apparatus according to inventive concept 217, wherein the chamber pressure stabilizer includes a mechanical user control element, which is arranged to set an axial position of the wider portion of the liquid column container with respect to the casing.
Inventive concept 221. The apparatus according to inventive concept 216, wherein at least an axial portion of the narrower portion of the liquid column container is flexible, so as to provide a variable axial length to the narrower portion.
Inventive concept 222. The apparatus according to inventive concept 221, wherein the at least an axial portion of the narrower portion of the liquid column container is elastic.
Inventive concept 223. The apparatus according to inventive concept 216, wherein at least an axial portion of the narrower portion of the liquid column container is telescopically adjustable, so as to provide a variable axial length to the narrower portion.
Inventive concept 224. The apparatus according to inventive concept 216, wherein the chamber pressure stabilizer includes a casing, and wherein the fluid reservoir is axially-slidably coupled to the casing.
Inventive concept 225. The apparatus according to inventive concept 196, wherein the chamber pressure stabilizer has a plurality of pressure indicia markings distributed along the liquid column container for measuring a height of the liquid in the liquid column container.
Inventive concept 226. The apparatus according to inventive concept 196, wherein the volume of the fluid reservoir is at least 3 cc.
Inventive concept 227. The apparatus according to inventive concept 226, wherein the volume of the fluid reservoir is at least 4 cc.
Inventive concept 228. The apparatus according to inventive concept 196, wherein the volume of the fluid reservoir is less than 5 cc.
Inventive concept 229. The apparatus according to inventive concept 196, wherein the liquid has a density of between 1.5 and 12 g/cm3 at 4 degrees Celsius at 1 atm.
Inventive concept 230. The apparatus according to any one of inventive concepts 257-271, wherein the liquid column container is open to the atmosphere at at least one site along the liquid column container.
Inventive concept 231. The apparatus according to inventive concept 230,

wherein the liquid column container has first and second ends at opposite ends of the liquid column container,

wherein the liquid column container is in fluid communication with the fluid reservoir via the first end, and

wherein the at least one site is at the second end, and the liquid column container is open to the atmosphere at the second end.
Inventive concept 232. The apparatus according to inventive concept 230, wherein the liquid column container defines an opening having an area of between 0.09 and 1 mm2, and wherein the liquid column container is open to the atmosphere via the opening.
Inventive concept 233. The apparatus according to inventive concept 232, wherein the cuff pressure stabilizer further includes a sealing element that is removably disposed so as to seal the opening.
Inventive concept 234. The apparatus according to any one of inventive concepts 257-271, wherein the chamber pressure stabilizer is arranged such that if the inflatable chamber is squeezed to a high threshold pressure, further squeezing of the inflatable chamber releases gas bubbles through the liquid in the liquid column container prevents the pressure from further increasing within the inflatable chamber.
Inventive concept 235. The apparatus according to any one of inventive concepts 257-271, wherein the inflatable chamber is an inflatable cuff, wherein the apparatus is for use with a tracheal ventilation tube having the inflatable cuff, the inflation lumen, and the inflation lumen proximal port, and wherein the inflation lumen proximal port connector is shaped to form the air-tight seal with the inflation lumen proximal port of the tracheal ventilation tube.
Inventive concept 236. The apparatus according to inventive concept 235, further including the tracheal ventilation tube, which includes the inflatable cuff, the inflation lumen, and the inflation lumen proximal port.
Inventive concept 237. The apparatus according to inventive concept 235, wherein the inflation lumen proximal port connector includes a male conical fitting with a taper.
Inventive concept 238. The apparatus according to inventive concept 237, wherein the taper is at least a 5% taper.
Inventive concept 239. The apparatus according to inventive concept 238, wherein the taper is a 6% taper, and the male conical fitting with the 6% taper complies with International Standard ISO 594-1:1986.

There is further provided, in accordance with an inventive concept 240 of the present invention, apparatus for use in contact with the atmosphere of the Earth and for use with a gas and a catheter having an inflatable cuff, an inflation lumen, and an inflation lumen proximal port, the apparatus including a cuff pressure stabilizer, which includes: an inflation lumen proximal port connector, which is shaped to form an air-tight seal with the inflation lumen proximal port of the catheter;

a gas inlet, which is in fluid communication with the inflation lumen proximal port connector;

a fluid reservoir, which has a volume of at least 2 cc, and which contains some of the gas;

a liquid column container, which is open to the atmosphere at at least one site along the liquid column container, and which is in fluid communication with the gas inlet via the fluid reservoir; and

a liquid, which is contained (a) in the fluid reservoir, (b) in the liquid column container, or (c) partially in the fluid reservoir and partially in the liquid column container,

wherein the cuff pressure stabilizer has a plurality of pressure indicia markings distributed along the liquid column container tube for measuring a height of the liquid in the liquid column container.
Inventive concept 241. The apparatus according to inventive concept 240, wherein the pressure indicia markings are distributed evenly throughout at least a relevant pressure range of 23-27 cm H2O.
Inventive concept 242. The apparatus according to inventive concept 240,

wherein the liquid column container has first and second ends at opposite ends of the liquid column container,

wherein the liquid column container is in fluid communication with the fluid reservoir via the first end, and

wherein the at least one site is at the second end, and the liquid column container is open to the atmosphere at the second end.
Inventive concept 243. The apparatus according to inventive concept 240, wherein the liquid has a density of between 1.5 and 12 g/cm3 at 4 degrees Celsius at 1 atm.
Inventive concept 244. The apparatus according to any one of inventive concepts 302-306, wherein the catheter is a tracheal ventilation tube, and wherein the apparatus is for use with the tracheal ventilation tube.
Inventive concept 245. The apparatus according to inventive concept 244, further including the tracheal ventilation tube, which includes the inflatable cuff, the inflation lumen, and the inflation lumen proximal port.
Inventive concept 246. The apparatus according to any one of inventive concepts 302-306, wherein the inflation lumen proximal port connector includes a male conical fitting with a taper.
Inventive concept 247. The apparatus according to inventive concept 246, wherein the taper is at least a 5% taper.
Inventive concept 248. The apparatus according to inventive concept 247, wherein the taper is a 6% taper, and the male conical fitting with the 6% taper complies with International Standard ISO 594-1:1986.

There is still further provided, in accordance with an inventive concept 249 of the present invention, apparatus for use in contact with the atmosphere of the Earth and for use with a gas and a catheter having an inflatable cuff, an inflation lumen, and an inflation lumen proximal port, the apparatus including a cuff pressure stabilizer, which includes:

an inflation lumen proximal port connector, which is shaped to form an air-tight seal with the inflation lumen proximal port of the catheter;

a gas inlet, which is in fluid communication with the inflation lumen proximal port connector;

a fluid reservoir, which has a volume of at least 2 cc, and which contains some of the gas;

a liquid column container, which is in fluid communication with the gas inlet via the fluid reservoir;

a liquid, which is contained (a) in the fluid reservoir, (b) in the liquid column container, or (c) partially in the fluid reservoir and partially in the liquid column container,

wherein the cuff pressure stabilizer has a plurality of pressure indicia markings distributed along the fluid column container for measuring a height of the liquid in the liquid column container, and

wherein the liquid column container is arranged such that, when (a) the liquid column container is oriented in an aligned orientation in which the pressure indicia markings reflect, to within 1 cm H2O, pressure of the gas in the fluid reservoir at least in a relevant pressure range of 23-27 cm H2O, and (b) the pressure of the gas in the fluid reservoir is 25 cm H2O:
an increase in a volume of the gas in the fluid reservoir of up to 2 cc results in less than a 10 cm H2O increase in the pressure of the gas in the fluid reservoir.
Inventive concept 250. The apparatus according to inventive concept 249, wherein the liquid column container is arranged such that, when (a) the liquid column container is oriented in the aligned orientation, and (b) the pressure of the gas in the fluid reservoir is 25 cm H2O:
   the increase in the volume of the gas in the fluid reservoir of up to 2 cc results in less than a 5 cm H2O increase in the pressure of the gas in the fluid reservoir.
Inventive concept 251. The apparatus according to inventive concept 249, wherein the liquid column container is arranged such that, when (a) the liquid column container is oriented in the aligned orientation, and (b) the pressure of the gas in the fluid reservoir is 25 cm H2O:
   a decrease in the volume of the gas in the fluid reservoir of up to 1 cc results in less than a 5 cm H2O decrease in the pressure of the gas in the fluid reservoir.
Inventive concept 252. The apparatus according to inventive concept 249, wherein the pressure indicia markings are distributed evenly throughout at least a relevant pressure range of 23-27 cm H2O.
Inventive concept 253. The apparatus according to any one of inventive concepts 312-315, wherein the inflation lumen proximal port connector includes a male conical fitting with a taper.
Inventive concept 254. The apparatus according to inventive concept 253, wherein the taper is at least a 5% taper.
Inventive concept 255. The apparatus according to inventive concept 254, wherein the taper is a 6% taper, and the male conical fitting with the 6% taper complies with International Standard ISO 594-1:1986.

There is additionally provided, in accordance with an inventive concept 256 of the present invention, apparatus for use on the Earth with a gas, the apparatus including:

a catheter, which includes an inflatable cuff, an inflation lumen, an inflation lumen proximal port, and an inflating tube, which couples the inflation lumen in fluid communication with the inflation lumen proximal port; and

a cuff pressure stabilizer, which includes:
an inflation lumen proximal port connector, which is shaped to form an air-tight seal with the inflation lumen proximal port of the catheter;
a gas inlet, which is in fluid communication with the inflation lumen proximal port connector;
a fluid reservoir, which has a volume of at least 2 cc, and which contains some of the gas;
a liquid column container, which is in fluid communication with the gas inlet via the fluid reservoir;
a liquid, which is contained (a) in the fluid reservoir, (b) in the liquid column container, or (c) partially in the fluid reservoir and partially in the liquid column container;
an inflation inlet port;
a first connector tube, which couples the inflation lumen proximal port connector in fluid communication with the inflation inlet port; and
a second connector tube, which couples the gas inlet in fluid communication with the inflation inlet port, such that the inflation lumen proximal port connector is in fluid communication with the gas inlet via the first connector tube and the second connector tube,

wherein the cuff pressure stabilizer has a plurality of pressure indicia markings distributed along the liquid column container for measuring a height of the liquid in the liquid column container, and

wherein, when (a) when the inflation lumen proximal port connector forms the air-tight seal with the inflation lumen proximal port of the catheter, and (b) a pressure of the gas in the fluid reservoir is 10 cm H2O:
(i) a combined air-flow resistance between the inflation inlet port and an interior of the inflatable cuff equals between 80% and 120% of (ii) a combined air-flow resistance between the inflation inlet port and the fluid reservoir.

There is yet additionally provided, in accordance with an inventive concept 257 of the present invention, a method for use in contact with the atmosphere of the Earth and for use with a gas and a catheter having an inflatable cuff, an inflation lumen, and an inflation lumen proximal port, the method including:

providing a cuff pressure stabilizer, which includes (a) an inflation lumen proximal port connector, which is shaped to form an air-tight seal with the inflation lumen proximal port of the catheter, (b) a fluid reservoir, (c) a liquid column container, which is (i) open to the atmosphere at at least one site along the liquid column container, (ii) in fluid communication with the fluid reservoir, and (iii) in communication with the inflation lumen proximal port connector via the fluid reservoir, and (d) a liquid, which is contained (i) in the fluid reservoir, (ii) in the liquid column container, or (iii) partially in the fluid reservoir and partially in the liquid column container, and which has a density of between 1.5 and 5 g/cm3 at 4 degrees Celsius at 1 atm, wherein the cuff pressure stabilizer has a plurality of pressure indicia markings distributed along the liquid column container for measuring a height of the liquid in the liquid column container; and

coupling the inflation lumen proximal port connector to the inflation lumen proximal port of the catheter.

There is further provided, in accordance with an inventive concept 258 of the present invention, a method for use in contact with the atmosphere of the Earth and for use with a gas and a catheter having an inflatable cuff, an inflation lumen, and an inflation lumen proximal port, the method including:

providing a cuff pressure stabilizer, which includes (a) an inflation lumen proximal port connector, which is shaped to form an air-tight seal with the inflation lumen proximal port of the catheter, (b) a gas inlet, which is in fluid communication with the inflation lumen proximal port connector, (c) a fluid reservoir, which has a volume of at least 2 cc, and which contains some of the gas, (d) a liquid column container, which is in fluid communication with the gas inlet via the fluid reservoir, and (e) a liquid, which is contained (i) in the fluid reservoir, (ii) in the liquid column container, or (iii) partially in the fluid reservoir and partially in the liquid column container, wherein the cuff pressure stabilizer has a plurality of pressure indicia markings distributed along the liquid column container for measuring a height of the liquid in the liquid column container;

coupling the inflation lumen proximal port connector to the inflation lumen proximal port of the catheter; and

orienting the liquid column container in an aligned orientation in which the pressure indicia markings reflect, to within 1 cm H2O, pressure of the gas in the fluid reservoir at least in a relevant pressure range of 23-27 cm H2O,

wherein when the liquid column container is oriented in the aligned orientation, the liquid column container has an inner cross-sectional area, measured in a horizontal plane, of at least 0.25 cm2 at a plurality of axial locations along the liquid column container corresponding to the pressure of the gas in the fluid reservoir at a respective plurality of pressures in the relevant pressure range.

There is still further provided, in accordance with an inventive concept 259 of the present invention, a method for use on the Earth with a gas and a catheter having an inflatable cuff, an inflation lumen, and an inflation lumen proximal port, the method including:

providing a cuff pressure stabilizer, which includes (a) an inflation lumen proximal port connector, which is shaped to form an air-tight seal with the inflation lumen proximal port of the catheter, (b) a gas inlet, which is in fluid communication with the inflation lumen proximal port connector, (c) a fluid reservoir, which has a volume of at least 2 cc, and which contains some of the gas, (d) a liquid column container, which is in fluid communication with the gas inlet via the fluid reservoir, and (e) a liquid, which is contained (i) in the fluid reservoir, (ii) in the liquid column container, or (iii) partially in the fluid reservoir and partially in the liquid column container, wherein the cuff pressure stabilizer has a plurality of pressure indicia markings distributed along the liquid column container for measuring a height of the liquid in the liquid column container;

coupling the inflation lumen proximal port connector to the inflation lumen proximal port of the catheter; and

orienting the liquid column container in an aligned orientation in which the pressure indicia markings reflect, to within 1 cm H2O, pressure of the gas in the fluid reservoir at least in a relevant pressure range of 23-27 cm H2O,

wherein when the liquid column container is oriented in the aligned orientation, an inner cross-sectional area of the liquid column container, measured in a horizontal plane, at a plurality of axial locations along the liquid column container corresponding to the pressure of the gas in the fluid reservoir at a respective plurality of pressures in the relevant pressure range is greater than the product of (a) a factor equal to 200% and (b) an average inner cross-sectional area of the liquid column container, measured in the horizontal plane, at all axial locations along the liquid column container corresponding to a pressure of the gas in the fluid reservoir of between 5 and 15 cm H2O.

There is additionally provided, in accordance with an inventive concept 260 of the present invention, a method for use in contact with the atmosphere of the Earth and for use with a gas, an inflatable chamber, an inflation lumen, and an inflation lumen proximal port in fluid communication with the inflatable chamber via the inflation lumen, the method including:

providing a chamber pressure stabilizer, which includes (a) an inflation lumen proximal port connector, which is shaped to form an air-tight seal with the inflation lumen proximal port, (b) a gas inlet, which is in fluid communication with the inflation lumen proximal port connector, (c) a fluid reservoir, which has a volume of at least 2 cc, and which contains some of the gas, (d) a liquid column container, which is in fluid communication with the gas inlet via the fluid reservoir, and which is shaped so as to define a wider portion and a narrower portion axially between the wider portion and the fluid reservoir, wherein the chamber pressure stabilizer is arranged to provide an adjustable distance between the wider portion of the liquid column container and the fluid reservoir, and (e) a liquid, which is contained (i) in the fluid reservoir, (ii) in the liquid column container, or (iii) partially in the fluid reservoir and partially in the liquid column container;

coupling the inflation lumen proximal port connector to the inflation lumen proximal port; and

adjusting the adjustable distance between the wider portion of the liquid column container and the fluid reservoir.
Inventive concept 261. The method according to inventive concept 260,

wherein the inflatable chamber is an inflatable cuff,

wherein the method is for use with a tracheal ventilation tube having the inflatable cuff, the inflation lumen, and the inflation lumen proximal port,

wherein the inflation lumen proximal port connector is shaped to form the air-tight seal with the inflation lumen proximal port of the tracheal ventilation tube, and

wherein coupling includes coupling the inflation lumen proximal port connector to the inflation lumen proximal port of the tracheal ventilation tube.

There is yet additionally provided, in accordance with an inventive concept 262 of the present invention, a method for use in contact with the atmosphere of the Earth and for use with a gas, an inflatable chamber, an inflation lumen, and an inflation lumen proximal port in fluid communication with the inflatable chamber via the inflation lumen, the method including:

providing a chamber pressure stabilizer, which includes (a) an inflation lumen proximal port connector, which is shaped to form an air-tight seal with the inflation lumen proximal port, (b) a gas inlet, which is in fluid communication with the inflation lumen proximal port connector, (c) a fluid reservoir, which has a volume of at least 2 cc, and which contains some of the gas, (d) a liquid column container, which is in fluid communication with the gas inlet via the fluid reservoir, and which is shaped so as to define a wider portion and a narrower portion axially between the wider portion and the fluid reservoir, (e) a liquid, which is contained (i) in the fluid reservoir, (ii) in the liquid column container, or (iii) partially in the fluid reservoir and partially in the liquid column container, wherein at each of all axial locations along the narrower portion of the liquid column container, the liquid column container (a) has a largest inner dimension equal to a greatest distance, in a plane perpendicular to a longitudinal axis of the liquid column container, between any two points within the liquid column container, and (b) can encompass a largest circle in the plane, and wherein at most or all of the axial locations along the narrower portion of the liquid column container, a ratio of (a) the largest inner dimension to (b) the diameter of the circle equals at least 2:1; and

coupling the inflation lumen proximal port connector to the inflation lumen proximal port.
Inventive concept 263. The method according to inventive concept 262,

wherein the inflatable chamber is an inflatable cuff,

wherein the method is for use with a tracheal ventilation tube having the inflatable cuff, the inflation lumen, and the inflation lumen proximal port,

wherein the inflation lumen proximal port connector is shaped to form the air-tight seal with the inflation lumen proximal port of the tracheal ventilation tube, and

wherein coupling includes coupling the inflation lumen proximal port connector to the inflation lumen proximal port of the tracheal ventilation tube.

There is also provided, in accordance with an inventive concept 264 of the present invention, a method for use in contact with the atmosphere of the Earth and for use with a gas and a catheter having an inflatable cuff, an inflation lumen, and an inflation lumen proximal port, the method including:

providing a cuff pressure stabilizer, which includes (a) an inflation lumen proximal port connector, which is shaped to form an air-tight seal with the inflation lumen proximal port of the catheter, (b) a gas inlet, which is in fluid communication with the inflation lumen proximal port connector, (c) a fluid reservoir, which has a volume of at least 2 cc, and which contains some of the gas, (d) a liquid column container, which is open to the atmosphere at at least one site along the liquid column container, and which is in fluid communication with the gas inlet via the fluid reservoir, and (e) a liquid, which is contained (i) in the fluid reservoir, (ii) in the liquid column container, or (iii) partially in the fluid reservoir and partially in the liquid column container, wherein the cuff pressure stabilizer has a plurality of pressure indicia markings distributed along the liquid column container tube for measuring a height of the liquid in the liquid column container; and

coupling the inflation lumen proximal port connector to the inflation lumen proximal port of the catheter.

There is further provided, in accordance with an inventive concept 265 of the present invention, a method for use in contact with the atmosphere of the Earth and for use with a gas and a catheter having an inflatable cuff, an inflation lumen, and an inflation lumen proximal port, the method including:

providing a cuff pressure stabilizer, which includes (a) an inflation lumen proximal port connector, which is shaped to form an air-tight seal with the inflation lumen proximal port of the catheter, (b) a gas inlet, which is in fluid communication with the inflation lumen proximal port connector, (c) a fluid reservoir, which has a volume of at least 2 cc, and which contains some of the gas, (d) a liquid column container, which is in fluid communication with the gas inlet via the fluid reservoir, and (e) a liquid, which is contained (i) in the fluid reservoir, (ii) in the liquid column container, or (iii) partially in the fluid reservoir and partially in the liquid column container, wherein the cuff pressure stabilizer has a plurality of pressure indicia markings distributed along the fluid column container for measuring a height of the liquid in the liquid column container;

coupling the inflation lumen proximal port connector to the inflation lumen proximal port of the catheter; and

orienting the liquid column container in an aligned orientation in which the pressure indicia markings reflect, to within 1 cm H2O, pressure of the gas in the fluid reservoir at least in a relevant pressure range of 23-27 cm H2O,

wherein the liquid column container is arranged such that, when (a) the liquid column container is oriented in the aligned orientation and (b) the pressure of the gas in the fluid reservoir is 25 cm H2O:
an increase in a volume of the gas in the fluid reservoir of up to 2 cc results in less than a 10 cm H2O increase in the pressure of the gas in the fluid reservoir.

There is still further provided, in accordance with an inventive concept 266 of the present invention, a method for use on the Earth with a gas, the method including:

inserting, into a trachea of a patient, a catheter, which includes an inflatable cuff, an inflation lumen, an inflation lumen proximal port, and an inflating tube, which couples the inflation lumen in fluid communication with the inflation lumen proximal port; and

providing a cuff pressure stabilizer, which includes (a) an inflation lumen proximal port connector, which is shaped to form an air-tight seal with the inflation lumen proximal port of the catheter, (b) a gas inlet, which is in fluid communication with the inflation lumen proximal port connector, (c) a fluid reservoir, which has a volume of at least 2 cc, and which contains some of the gas, (d) a liquid column container, which is in fluid communication with the gas inlet via the fluid reservoir, (e) a liquid, which is contained (i) in the fluid reservoir, (ii) in the liquid column container, or (iii) partially in the fluid reservoir and partially in the liquid column container, (f) an inflation inlet port, (g) a first connector tube, which couples the inflation lumen proximal port connector in fluid communication with the inflation inlet port, and (h) a second connector tube, which couples the gas inlet in fluid communication with the inflation inlet port, such that the inflation lumen proximal port connector is in fluid communication with the gas inlet via the first connector tube and the second connector tube, wherein the cuff pressure stabilizer has a plurality of pressure indicia markings distributed along the liquid column container for measuring a height of the liquid in the liquid column container; and

coupling the inflation lumen proximal port connector to the inflation lumen proximal port of the catheter,

wherein when (a) the inflation lumen proximal port connector forms the air-tight seal with the inflation lumen proximal port of the catheter, and (b) a pressure of the gas in the fluid reservoir is 10 cm H2O:
(i) a combined air-flow resistance between the inflation inlet port and an interior of the inflatable cuff equals between 80% and 120% of (ii) a combined air-flow resistance between the inflation inlet port and the fluid reservoir.

The present invention will be more fully understood from the following detailed description of embodiments thereof, taken together with the drawings, in which:

### BRIEF DESCRIPTION OF THE DRAWINGS

FIG. 1 is a schematic illustration of a cuff pressure stabilizer for use with a tracheal ventilation tube, in accordance with an application of the present invention;

FIG. 2 is a schematic illustration of another cuff pressure stabilizer for use with a tracheal ventilation tube, in accordance with an application of the present invention;

FIGS. 3A-B are additional views of the cuff pressure stabilizer of FIG. 2, in accordance with an application of the present invention;

FIG. 4 is a schematic illustration of a yet another cuff pressure stabilizer for use with a tracheal ventilation tube, in accordance with an application of the present invention;

FIGS. 5A and 5B are schematic illustrations of the cuff pressure stabilizer of FIG. 4 adjusted in respective different states, in accordance with an application of the present invention;

FIG. 6 is another schematic illustration of the cuff pressure stabilizer of FIG. 4, in accordance with an application of the present invention;

FIG. 7 is a cross-sectional view of the cuff pressure stabilizer of FIG. 6 taken along the line VII-VII of FIG. 6, in accordance with an application of the present invention;

FIG. 8 is a schematic illustration of another cuff pressure stabilizer for use with a tracheal ventilation tube, in accordance with an application of the present invention;

FIG. 9 is a cross-sectional view of the cuff pressure stabilizer of FIG. 8 taken along the line IX-IX of FIG. 8, in accordance with an application of the present invention;

FIGS. 10A-B are schematic illustrations of the cuff pressure stabilizer of FIG. 8 in resting and pressurized states, respectively, in accordance with an application of the present invention;

FIGS. 11A-B are schematic illustrations of yet another cuff pressure stabilizer in resting and pressurized states, respectively, for use with a tracheal ventilation tube, in accordance with an application of the present invention;

FIGS. 12A-B and 13A-B are schematic illustrations of still another cuff pressure stabilizer in resting and pressurized states, respectively, for use with a tracheal ventilation tube, in accordance with an application of the present invention;

FIGS. 14A-B are schematic illustrations of yet another cuff pressure stabilizer in resting and pressurized states, respectively, for use with a tracheal ventilation tube, in accordance with an application of the present invention; and

FIGS. 15A-B are photographs of prototypes of the cuff pressure stabilizer of FIGS. 12A-B and 13A-B, in accordance with respective applications of the present invention.

### DETAILED DESCRIPTION OF APPLICATIONS

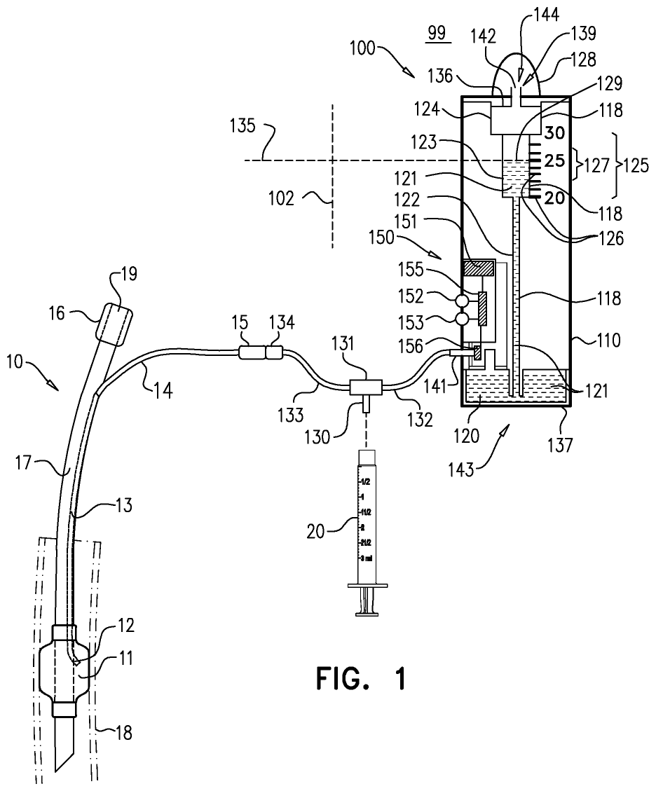

FIG. 1 is a schematic illustration of a cuff pressure stabilizer 100 for use with a catheter, such as a tracheal ventilation tube 10, in accordance with an application of the present invention. Cuff pressure stabilizer 100 is for use in contact with the atmosphere 99 (i.e., ambient air) of the Earth. Cuff pressure stabilizer 100 is for use with a gas, typically air.

Tracheal ventilation tube 10 comprises an inflatable cuff 11, an inflation lumen 13, and an inflation lumen proximal port 15. Inflatable cuff 11 may comprise, for example, a balloon, and is typically mounted on tracheal ventilation tube 10 near a distal end of the tracheal ventilation tube, e.g., within 3 cm, such as within 1 cm, of the distal end. Inflatable cuff 11 typically comprises a nearly non-compliant material. A “balloon,” as used in the present application, including the claims, is an inflatable flexible bag, having any level of elasticity, including nearly non-elastic. Typically, inflatable cuff 11 has a volume of around 10 cc. Tracheal ventilation tube 10 typically further comprises a cuff inflation lumen distal port 12, a tracheal ventilation tube ventilation port 16, a tracheal ventilation tube ventilation lumen 17, and a tracheal ventilation tube ventilator connection 19. For some applications, tracheal ventilation tube 10 further comprises an inflating tube 14, which couples inflation lumen 13 in fluid communication with inflation lumen proximal port 15. Tracheal ventilation tube 10 is schematically shown inserted into a trachea 18. Inflatable cuff 11 is inflatable into sealing contact with the inner surface of trachea 18. As used in the present application, including in the claims, a “tracheal ventilation tube” comprises an endotracheal tube (ETT) or a tracheostomy tube.

Cuff pressure stabilizer 100 comprises:
a casing 110;
an inflation lumen proximal port connector 134, which is shaped to form an air-tight seal with inflation lumen proximal port 15 of tracheal ventilation tube 10;
a gas inlet 141, which is in fluid communication with inflation lumen proximal port connector 134;
a fluid reservoir 120, which typically has a volume of at least 2 cc (such as at least 3 cc, e.g., at least 4 cc), and optionally has a volume of less than 5 cc, and which contains some of the gas;
a liquid column container 118, which is in fluid communication with gas inlet 141 via fluid reservoir 120; and
a liquid 121, which is contained (a) in fluid reservoir 120, (b) in liquid column container 118, or (c) partially in fluid reservoir 120 and partially in liquid column container 118.

As can be seen, the gas partially fills fluid reservoir 120. When the system is in equilibrium, the pressure of the gas in fluid reservoir 120 equals the pressure of the gas in inflatable cuff 11. For some applications, inflation lumen proximal port connector 134 comprises a male conical fitting with a taper. For some applications, the taper is at least a 5% taper. For some applications, the taper is a 6% taper, and the male conical fitting with the 6% taper complies with International Standard ISO 594-1:1986, which is the standard for connections to conventional inflation lumen proximal ports of tracheal ventilation tubes. Typically, gas inlet 141 has a large cross-sectional area, such as at least 9 mm2, so that any droplets of liquid 121 that should happen to form will not clog the gas inlet.

Cuff pressure stabilizer 100 has a plurality of pressure indicia markings 126 distributed along liquid column container 118 for measuring a height of liquid 121 in liquid column container 118. Typically, casing 110 has pressure indicia markings 126, as shown in the figures, in which case pressure indicia markings 126 are distributed along liquid column container 118 by being distributed alongside liquid column container 118. Alternatively, for some applications, liquid column container 118 has pressure indicia markings 126 therealong. Liquid column container 118 is used in an aligned orientation (hereinbelow, the “aligned orientation”) in which pressure indicia markings 126 reflect, to within 1 cm H2O (i.e., with no error or an error of no more than 1 cm H2O), pressure of the gas in fluid reservoir 120 at least in a relevant pressure range 127 of 23-27 cm H2O, such as a range of 22-28 cm H2O, e.g., a range of 20-30 cm H2O, as marked numerically in the figures. The full marked scale 125 range typically extends both above and below the range of 20-30 cm H2O. The pressure is read by comparing a level 129 of fluid in liquid column container 118 with pressure indicia markings 126, as is known in the manometer art.

Typically, pressure indicia markings 126 reflect the height of liquid 121 in liquid column container 118 relative to the height of liquid 121 in fluid reservoir 120, as is known in the manometer art (as the liquid surface ascends in liquid column container 118, the liquid surface descends in fluid reservoir 120, though typically not by the same changes in heights). Therefore, the spacing of pressure indicia markings 126 depends in part on the shape and volume of fluid reservoir 120. For example, the spacing of pressure indicia markings 126 between 25 and 26 cm H2O may be different from the spacing of pressure indicia markings 126 between 26 and 27 cm H2O. In addition, for example, the wider the fluid reservoir, the greater the spacing of pressure indicia markings 126. For some applications, pressure indicia markings 126 are distributed evenly throughout at least relevant pressure range 127.

Typically, for applications in which liquid 121 comprises water, pressure indicia markings 126 are spaced at close to 1-cm intervals. For applications in which liquid 121 comprises a liquid with a higher or lower density than that of water, pressure indicia markings 126 are not spaced at 1-cm intervals, such as described hereinbelow. Optionally, liquid 121 comprises a dye to increase the visibility of the liquid for making the pressure measurements.

Typically, liquid column container 118 is configured to automatically assume the aligned orientation when cuff pressure stabilizer 100 is hung from or otherwise attached to a conventional IV pole, hospital wall, or other surface or object. For example, cuff pressure stabilizer 100 may comprise a coupling element that is configured to automatically orient liquid column container 118 in the aligned orientation. The coupling element may comprise a hook or a loop 128 that is hangable from a conventional hook of a conventional IV pole, similar to the standard hook of IV bags. Alternatively or additionally, the coupling element may comprise a squeezing coupler (e.g., a gripper or a clamp) that is coupleable to a vertical pole (e.g., a vertical IV pole) or a horizontal pole (e.g., a horizontal portion of one of the hooks of the IV pole), or another connector that is configured to be attached to a vertical surface, such as a hospital wall.

For some applications, when liquid column container 118 is oriented in the aligned orientation, liquid column container 118 has an inner cross-sectional area, measured in a horizontal plane 135, of at least 0.16 cm2 (e.g., at least 0.25 cm2, 0.5 cm2, or 1 cm2) at a plurality of (such as at most or all) axial locations along liquid column container 118 corresponding to the pressure of the gas in fluid reservoir 120 at a respective plurality of pressures in relevant pressure range 127. Typically, the inner cross-sectional area, measured in horizontal plane 135, is less than 2 cm2 at a plurality of (such as at most or all) axial locations along liquid column container 118 corresponding to the pressure of the gas in fluid reservoir 120 at a respective plurality of pressures in relevant pressure range 127, when liquid column container 118 is oriented in the aligned orientation.

As a result of this relatively large cross-sectional area, cuff pressure stabilizer 100 regulates (i.e., reduces fluctuations) the pressure of the gas in fluid reservoir 120 at least for changes of gas volume in the range of 0-2 cc, and thus at gas inlet 141 and in inflatable cuff 11, in addition to measuring the pressure. In contrast, conventional manometers only measure the pressure, without substantially affecting the pressure, as it is ideally and commonly the goal of measurement devices to not affect the measured target. For a given cuff of initial gas volume V, as the squeezing of inflatable cuff 11 by trachea 18 increases such that the available gas volume decreases, the volume of the gas in inflatable cuff 11 decreases by some fraction equal to the change in V divided by V because the cuff is nearly non-compliant. For endotracheal tubes without external regulation, this decrease in volume of the inflatable cuff results in an increase in pressure of the gas within the system, including within the inflatable cuff, since the gas contained in the cuff has no significant external volume to move into, in accordance with the ideal gas law; the opposite occurs when the level of squeezing on the cuff by the trachea decreases.

In experiments conducted by the inventors, the inventors found that, for real endotracheal tube cuff balloons of volumes around 10 cc, each 0.1 cc decrease in volume in the inflatable cuff resulted in about a 1 cm H2O increase in pressure in the system and the cuff, and each 0.1 cc increase in volume in the inflatable cuff resulted in about a 1 cm H2O decrease in pressure in the system and the cuff. This is a surprising significant departure from the ideal non-compliant gas law calculation which would predict a 0.01 cc volume change per 1 cm H2O pressure change. The inventors thus concluded that real endotracheal tube cuff balloons are in fact semi-compliant. Therefore, the mitigation volumes should be calculated based on the experimental finding. In clinical practice, the pressure in ETT inflatable cuffs generally varies +/−10 cm H2O from the typically target pressure of 25 cm H2O, i.e., varies between 15 and 35 cm H2O. Based on the above-mentioned experimental data, the inventors appreciated that the volume in ETT inflatable cuffs generally varies by +/−1 cc (+/−10 cm H2O times 0.1 cc/cm H2O), i.e., a total range of 2 cc, and, among a broader spectrum of patients, the volume of ETT inflatable cuffs generally varies by +/−20 cm H2O from the typical target pressure of 25 cm H2O, i.e., a total range of at least 4 cc.

The inventors appreciated that to the extent that cuff pressure stabilizer 100 is able to offset the changes in volume in inflatable cuff 11, the pressure changes are also offset, thereby stabilizing the pressure in inflatable cuff 11. Cuff pressure stabilizer 100 is able to offset the changes in volume in inflatable cuff 11 because of the relatively large cross-sectional area of liquid column container 118 at relevant pressure range 127, e.g., 23-27 cm H2O.

For example, assume that (a) liquid 121 comprises water, (b) the cross-sectional area of liquid column container 118 at relevant pressure range 127 is 1 cm2, (c) pressure indicia markings 126 are spaced at 1-cm intervals, and (d) the initial pressure in inflatable cuff 11 is 25 cm H2O. A decrease in volume of inflatable cuff 11 of 1 cc (caused by increased squeezing by the trachea) would displace from the inflatable cuff the excess 1 cc of gas into fluid reservoir 120, and a corresponding additional 1 cc of water out of fluid reservoir 120 into liquid column container 118. This additional 1 cc of water would fill an additional 1 cc of fluid reservoir 120, raising level 129 of fluid by 1 cm, and thus the pressure in inflatable cuff 11 (as indicated by pressure indicia markings 126) by 1 cm H2O, from 25 cm H2O to 26 cm H2O.

For a real inflatable cuff having a volume of 10 cc without attachment of the regulation system, a decrease in volume of inflatable cuff 11 of 1 cc would have resulted in an increase of the cuff pressure gas by about 10 cm H2O, based on the inventors' experimental data, i.e., the integration of the pressure regulator with tracheal ventilation tube 10 results in a factor of 10 suppression of the pressure change, resulting in the pressure regulation described herein.

More generally, the change in pressure in inflatable cuff 11 within relevant pressure range 127, resulting from a change in volume of inflatable cuff 11, when liquid 121 does not necessarily comprise water, is expressed by the following Equation 1:

ΔP=(ΔV/A)*d

in which:
ΔV is the change in volume in cc of inflatable cuff 11,
d is the density of liquid 121 in g/cm3 at 4 degrees Celsius at 1 atm,
ΔP is the change in pressure in cm H2O in inflatable cuff 11, and
A is the average cross-sectional area in cm2 of liquid column container 118 along the axial portion of the liquid column container in which the change in liquid height occurs during the change in volume of the inflatable cuff.

As used in the present application, including in the claims, “horizontal” means horizontal with respect to the Earth, i.e., perpendicular to a vertical line 102 directed to the center of gravity of the Earth, e.g., as ascertained using a plumb-line.

For some applications, at the plurality of axial locations along liquid column container 118 corresponding to the pressure of the gas in fluid reservoir 120 at the respective plurality of pressures in relevant pressure range 127, the inner cross-sectional area is at least 0.16 cm2 (e.g., at least 0.25 cm2, 0.5 cm2, or 1 cm2), when liquid column container 118 is oriented in the aligned orientation.

Typically, liquid column container 118 is wider (i.e., has a greater cross-sectional area) in (a) a relevant-range fluid compartment 123 that includes relevant pressure range 127 than in (b) a lower-range fluid compartment 122 that reflects pressures of the gas in fluid reservoir 120 of less than 20 cm H2O. In other words, when liquid column container 118 is oriented in the aligned orientation, an average inner cross-sectional area, measured in horizontal plane 135, at all axial locations along liquid column container 118 corresponding to the pressure of the gas in fluid reservoir 120 at the respective plurality of pressures in relevant pressure range 127 is greater than the product of (a) a factor greater than one and (b) an average inner cross-sectional area of liquid column container 118, measured in horizontal plane 135, at all axial locations along liquid column container 118 corresponding to pressure of the gas in fluid reservoir 120 of less than 20 cm H2O. For some applications, the factor is 150%, such as 200%, 300%, 400%, or 500%. The narrower liquid column container 118 in lower-range fluid compartment 122 reduces the total required amount of liquid 121, which is useful in configurations in which liquid 121 comprises an expensive heavy liquid.

For some of these applications, when liquid column container 118 is oriented in the aligned orientation, the inner cross-sectional area at the plurality of axial locations along liquid column container 118 corresponding to the pressure of the gas in fluid reservoir 120 at the respective plurality of pressures in relevant pressure range 127 equals at least 200% of an average inner cross-sectional area of liquid column container 118, measured in horizontal plane 135, at most or all axial locations along liquid column container 118 corresponding to a pressure of the gas in fluid reservoir 120 of between 5 and 15 cm H2O. For some applications, the average inner cross-sectional area is less than 0.16 cm2, such as less than 0.09 cm2, at most or all axial locations along liquid column container 118 corresponding to a pressure of the gas in fluid reservoir 120 of between 5 and 15 cm H2O, when liquid column container 118 is oriented in the aligned orientation.

For some applications, liquid column container 118 is wider (i.e., has a greater cross-sectional area) in (a) a buffer fluid compartment 124 that reflects pressures of the gas in fluid reservoir 120 of greater than 28 cm H2O, e.g., greater than 30 cm H2O that includes relevant pressure range 127 than in (b) relevant-range fluid compartment 123 that includes relevant pressure range 127. In other words, when liquid column container 118 is oriented in the aligned orientation, an average inner cross-sectional area at all axial locations along liquid column container 118 corresponding to the pressure of the gas in fluid reservoir 120 at the respective plurality of pressures in the relevant range is less than the inner cross-sectional area of liquid column container 118, measured in horizontal plane 135, at at least one axial location along liquid column container 118 corresponding to a pressure of the gas in fluid reservoir 120 of between 28 cm and 35 cm H2O. The wider liquid column container 118 in buffer fluid compartment 124 substantially reduces increases in pressure if the pressure should exceed the lower end of the pressure range of buffer fluid compartment 124, because buffer fluid compartment 124 can hold a greater volume of liquid per unit of height than can relevant-range fluid compartment 123.

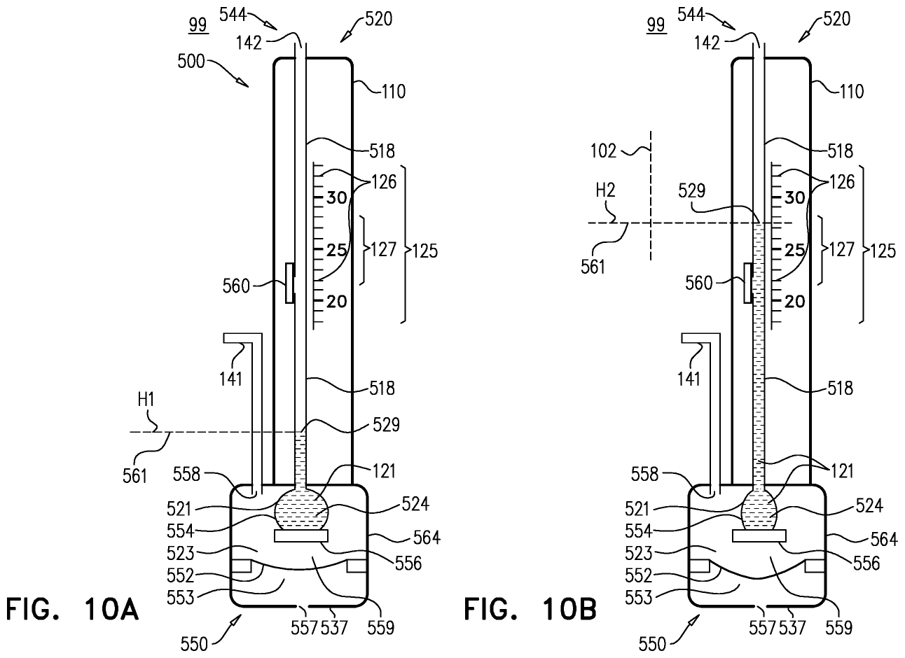

For some applications, liquid 121 has a density of between 0.8 and 12 g/cm3 at 4 degrees Celsius at 1 atm, and/or a density of between 0.8 and 12 g/cm3 at 20 degrees Celsius at 1 atm. Typically, the density (whether at 4 degrees or at 20 degrees) is between 1.5 and 5 g/cm3, such as between 2 and 4 g/cm3, e.g., between 2.5 and 3.5 g/cm3 (all of these values are more dense than water and less dense than mercury). For some applications, liquid 121 comprises a tungstate-based liquid, e.g., selected from the group consisting of: sodium polytungstate, sodium metatungstate, lithium polytungstate, and lithium metatungstate. Alternatively or additionally, liquid 121 may have any of the characteristics described hereinbelow with reference to FIG. 8.

To the extent that the density d of liquid 121 is greater than that of water, i.e., greater than 1 g/cm3 at 4 degrees Celsius at 1 atm, a shorter liquid column container 118 can be used to measure and regulate pressures, and pressure indicia markings 126 are closer together. Assuming a container of uniform cross section and a liquid column of uniform cross section, it follows from Equation 1 (ΔP=(ΔV/A)*d) that, if using a liquid of density d compared with using water, the distance between pressure indicia markings 126 for indicating a 1 cm H2O change in pressure equals the quotient of (a) 1 cm divided by (b) the density of liquid 121 at 4 degrees Celsius at 1 atm. For some applications, a distance between a highest point 136 of liquid column container 118 and a lowest point 137 of fluid reservoir 120 is between 10 and 20 cm, when liquid column container 118 is oriented in an aligned orientation. The highest point is measured with respect to the center of gravity of the Earth.

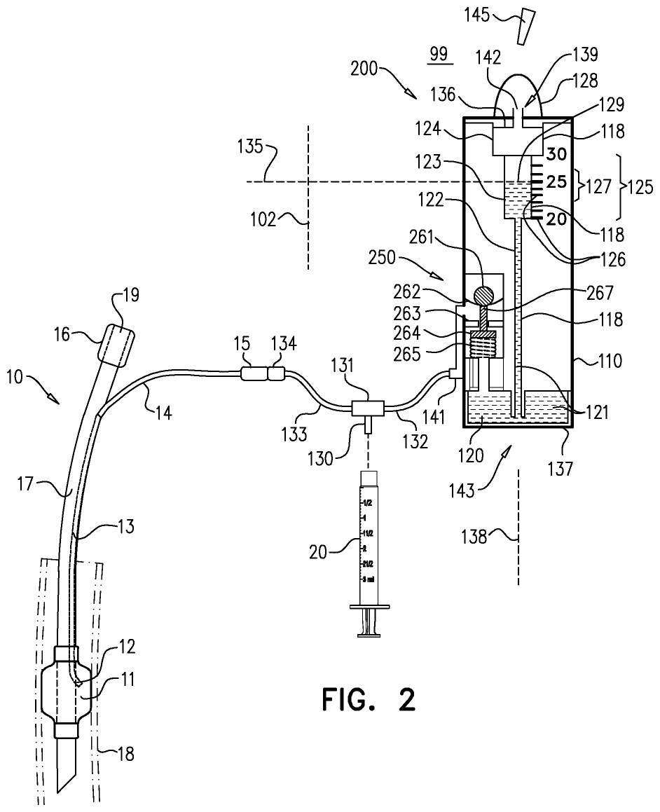

For some applications, as shown in the figures and labeled in FIG. 2 (described hereinbelow), a central longitudinal axis 138 of liquid column container 118 is perpendicular to horizontal plane 135, when liquid column container 118 is oriented in the aligned orientation. For other applications, central longitudinal axis 138 of liquid column container 118 is not perpendicular to horizontal plane 135, when liquid column container 118 is oriented in the aligned orientation (configuration not shown).

Typically, liquid column container 118 is open to atmosphere 99 at at least one site 139 along liquid column container 118. Liquid column container 118 has first and second ends 143 and 144 at opposite ends of liquid column container 118. For some applications, liquid column container 118 is in fluid communication with fluid reservoir 120 via first end 143, the at least one site 139 is at second end 144, and liquid column container 118 is open to atmosphere 99 at second end 144. For some applications, liquid column container 118 defines an opening 142 having an area of between 0.09 and 1 mm2, and liquid column container 118 is open to atmosphere 99 via opening 142. For some applications, cuff pressure stabilizer 100 further comprises a sealing element 145 (e.g., a plug or screw-cap) that is removably disposed so as to seal opening 142 (shown removed in FIG. 2). Alternatively or additionally, for some applications, cuff pressure stabilizer 100 further comprises a container-sealing element that is removably disposed between fluid reservoir 120 and liquid column container 118 so as to prevent fluid communication between fluid reservoir 120 and liquid 121 container column (configuration not shown).

For some applications, when liquid column container 118 is oriented in the aligned orientation, at least 2 cc, no more than 10 cc, and/or between 2 and 10 cc (e.g., between 2 and 8 cc, such as between 2 and 6 cc, e.g., between 2 and 4 cc) of liquid 121 are contained in fluid reservoir 120 at a lower height than first end 143 of liquid column container 118. For some applications, an upper surface area of liquid 121 in fluid reservoir 120 is at least 2 cm2, no more than 8 cm2, and/or between 2 and 8 cm2 (e.g., between 2 and 6 cm2, such as between 2 and 4 cm2), when (a) liquid column container 118 is oriented in the aligned orientation and (b) the pressure of the gas in fluid reservoir 120 is 25 cm H2O.

For some applications, cuff pressure stabilizer 100 further comprises:
an inflation inlet port 130, which is coupleable with an external inflation source 20, such as a syringe;
a first connector tube 133, which couples inflation lumen proximal port connector 134 in fluid communication with inflation inlet port 130; and
a second connector tube 132, which couples gas inlet 141 in fluid communication with inflation inlet port 130, such that inflation lumen proximal port connector 134 is in fluid communication with gas inlet 141 via first connector tube 133 and second connector tube 132.

Typically, inflation inlet port 130 comprises a valve, such as a directional valve. Inflation inlet port 130 isolates the system such there is no exchange of gas (air) between inflatable cuff 11 and atmosphere 99 (ambient air) after initial inflation by external inflation source 20.

For some applications, cuff pressure stabilizer 100 further comprises an inlet junction 131, which comprises inflation inlet port 130, and which couples in fluid communication inflation inlet port 130, first connector tube 133, and second connector tube 132.

For some applications, when (a) inflation lumen proximal port connector 134 forms the air-tight seal with inflation lumen proximal port 15 of tracheal ventilation tube 10 and (b) a pressure of the gas of the gas in fluid reservoir 120 is 10 cm H2O, (i) a first combined air-flow resistance between inflation inlet port 130 and an interior of inflatable cuff 11 equals between 80% and 120% of (ii) a second combined air-flow resistance between inflation inlet port and fluid reservoir 120, such as between 90% and 110%, e.g., between 95% and 105%. Typically, in order to achieve these relative air-flow resistances, the relative lengths of first and second connector tubes 133 and 132 are set such that the resistance of second connector tube 132 equals the sum of the resistance of first connector tube 133 and a fixed constant resistance of all elements of tracheal ventilation tube 10 in the flow path. This approximately equal air-flow resistance prevents transient false pressure readings immediately following inflation or reinflation of inflatable cuff 11 via inflation inlet port 130, without being dependent on the technique of the healthcare worker. For example, if the resistance from inflation inlet port 130 were lower in second connector tube 132 (to cuff pressure stabilizer 100) than in first connector tube 133 (to inflatable cuff 11), during inflation initially a majority of the air would flow toward cuff pressure stabilizer 100. As a result, level 129 of fluid in liquid column container 118 would indicate a higher pressure than the true pressure of inflatable cuff 11. If external inflation source 20 were to be disconnected at this point in time, the pressure shown by liquid column container 118 would gradually decrease as pressure equilibrium between inflatable cuff 11 and fluid reservoir 120 is gradually reached.

For some applications, cuff pressure stabilizer 100 further comprises one or more connector tubes, which couple inflation lumen proximal port connector 134 in fluid communication with gas inlet 141. When inflation lumen proximal port connector 134 forms the air-tight seal with inflation lumen proximal port 15 of tracheal ventilation tube 10, a combined air-flow resistance between an interior of inflatable cuff 11 and fluid reservoir 120 is such that a transient pressure difference of 5 cm H2O between the interior of inflatable cuff 11 and fluid reservoir 120 results in less than a 0.1 cc per second, e.g., less than a 0.05, a 0.02, or a 0.01 cc per second, fluid flow from inflatable cuff 11 into fluid reservoir 120. The slow rate of flow delays the automatic pressure-regulation response from cuff pressure stabilizer 100. A too rapid pressure-regulation response might underinflate inflatable cuff 11 during transient, short-term increases in pressure in the inflatable cuff, such as during the positive pressure phase of the ventilation cycle when high-pressure ventilation (generally greater than 25 cm H2O) is applied to the patient, generally for only a few seconds, typically less than 3 seconds.

For patients ventilated at high peak inspiratory pressure (PIP), i.e., greater than 25 cm H2O and sometimes even up to 40 cm H2O, there is a need to both maintain the high ventilation pressure during the peak ventilation and to maintain on average the balloon pressure near 25 cm H2O. For these patients only, it is advantageous to limit the fluid flow between inflatable cuff 11 and fluid reservoir 120, as described immediately above, at the expense of increasing the pressure regulation response time of cuff pressure stabilizer 100.

Generally, high PIP is applied to less than 30% of patients. For the remaining 70% of patients there is no need for flow limitation. To the contrary, the longer response time due to flow limitation compromises the desired fast response to low cuff pressures. To best accommodate these differing patient needs, in some applications, cuff pressure stabilizer 100 comprises a switch that sets flow states of cuff pressure stabilizer 100, including (a) a flow-limiting state (e.g., via a flow-limiting channel, as described immediately above, and (b) a fast-flow state (e.g., via a fast-flow channel, which is sized so as to substantially not limit flow, e.g., such that a transient pressure difference of 5 cm H2O between the interior of inflatable cuff 11 and fluid reservoir 120 results in greater than a 0.02 cc per second, e.g., more than a 0.05 cc per second, fluid flow from inflatable cuff 11 into fluid reservoir 120). A healthcare worker selects which of these two channels to enable according to the individual patient's ventilation needs. In addition, the healthcare worker may select the fast-flow channel during inflation of inflatable cuff 11.

For some applications, the flow resistance is placed not between inflatable cuff 11 and fluid reservoir 120, but instead at the at least one site 139 along liquid column container 118 at which liquid column container 118 is open to atmosphere 99, as described hereinabove. Flow resistance anywhere along the fluid communication from inflatable cuff 11 all the way to atmosphere 99 is sufficient to create the desired effect.

For some applications, liquid column container 118 is arranged such that, when (a) liquid column container 118 is oriented in the aligned orientation and (b) the pressure of the gas in fluid reservoir 120 is 25 cm H2O: an increase in a volume of the gas in fluid reservoir 120 of up to 2 cc results in less than a 10 cm H2O increase in the pressure of the gas in fluid reservoir 120, such as less than a 6 cm H2O increase in the pressure of the gas in fluid reservoir 120, e.g., less than a 5 cm H2O or less than a 4 cm H2O increase. Alternatively or additionally, for some applications, liquid column container 118 is arranged such that, when (a) liquid column container 118 is oriented in the aligned orientation, and (b) the pressure of the gas in fluid reservoir 120 is 25 cm H2O: a decrease in the volume of the gas in fluid reservoir 120 of up to 1 cc results in less than a 6 cm H2O decrease in the pressure of the gas in fluid reservoir 120, e.g., less than a 5 cm H2O or less than a 4 cm H2O decrease. For some applications, liquid column container 118 is shaped so as to provide asymmetric regulation of pressure; for example, liquid column container 118 may be conical. Alternatively or additionally, for some applications, pressure indicia markings 126 are arranged to indicate a pressure of 25 cm H2O at an axial location of relevant-range fluid compartment 123 other than an axial center of relevant-range fluid compartment 123.

Typically, cuff pressure stabilizer 100 does not comprise any membranes in contact with liquid 121, and does not comprise any membranes in a fluid path between liquid 121 and atmosphere 99.

Typically, cuff pressure stabilizer 100 does not comprise a spring for measuring the pressure of the gas in fluid reservoir 120.

For some applications, cuff pressure stabilizer 100 further comprises an orientation-sensitive valve assembly 150, which comprises a valve 156 (e.g., a solenoid valve, or an elastically biased gate). Orientation-sensitive valve assembly 150 is arranged to automatically assume:
an open state when an orientation of cuff pressure stabilizer 100 differs from the aligned orientation by no more than a constant number of degrees, and
a reduced-flow state when the orientation of cuff pressure stabilizer 100 differs from the aligned orientation by more than the constant number of degrees.

Typically, the constant equals between 5 and 45 degrees, such as between 5 and 20 degrees.

Valve 156 is configured to reduce fluid communication thereacross by at least 90% when in the reduced-flow state compared to when in the open state, such as to entirely block fluid communication thereacross when in the reduced-flow state. Such reduced fluid communication serves as a safety feature and/or to prevent spillage of the fluid during storage and shipment of the device. For some applications, valve 156 is arranged in a fluid path between inflation lumen proximal port connector 134 and fluid reservoir 120.

For some applications, orientation-sensitive valve assembly 150 comprises electronic components, such as an orientation sensor 155 (e.g., comprising an accelerometer), which is configured to sense the orientation of cuff pressure stabilizer 100, and a battery 151. For some applications, orientation-sensitive valve assembly 150 further comprises one or more alignment indicators 152 and 153 (e.g., LEDs).

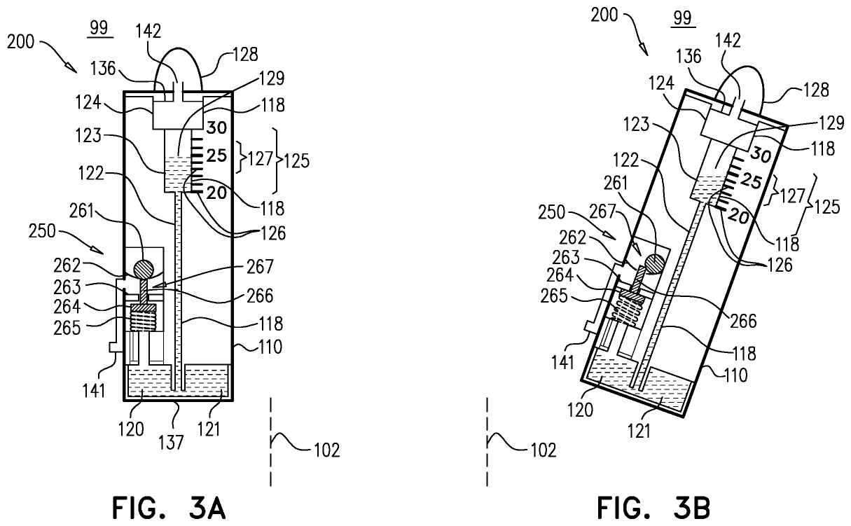

Reference is now made to FIGS. 2 and 3A-B, which are schematic illustrations of a cuff pressure stabilizer 200 for use with tracheal ventilation tube 10, in accordance with an application of the present invention. Except as described below, cuff pressure stabilizer 200 is identical to cuff pressure stabilizer 100, described hereinabove with reference to FIG. 1.

For some applications, cuff pressure stabilizer 200 comprises an orientation-sensitive valve assembly 250 that is mechanical and non-electrical. For some applications, orientation-sensitive valve assembly 250 comprises:
a moving weight 261, which is typically spherical;
a curved sliding/rolling surface 262, which is shaped to define an opening 267 therethrough at its bottom (when liquid column container 118 is oriented in the aligned orientation), the opening too small for moving weight 261 to pass through;
a gas passage opening 263;
a seal 264, which is shaped and arranged to seal gas passage opening 263 when seal 264 is up with respect to the Earth;
an elastic element 265 (e.g., a spring), which is arranged to push seal 264 against gas passage opening 263; and
a press-switch 266, which has (a) a first end that is fixed to seal 264 and passes through gas passage opening 263, and a (b) second end that passes through opening 267.

When cuff pressure stabilizer 100 differs from the aligned orientation by more than the constant number of degrees mentioned above with reference to FIG. 1 regarding orientation-sensitive valve assembly 150, moving weight 261 slides or rolls over opening 267, such that moving weight 261 presses on the second end of press-switch 266, such as shown in FIG. 3A. As a result, press-switch 266 pushes seal 264 away from gas passage opening 263 and compresses elastic element 265, thereby allowing gas passage through the gas passage opening and opening the valve.

When cuff pressure stabilizer 100 differs from the aligned orientation by no more than the constant number of degrees, moving weight 261 slides or rolls away from opening 267, such that moving weight 261 does not press on the second end of press-switch 266, such as shown in FIG. 3B. As a result, elastic element 265 pushes seal 264 against gas passage opening 263, thereby blocking the gas passage opening and closing the valve.

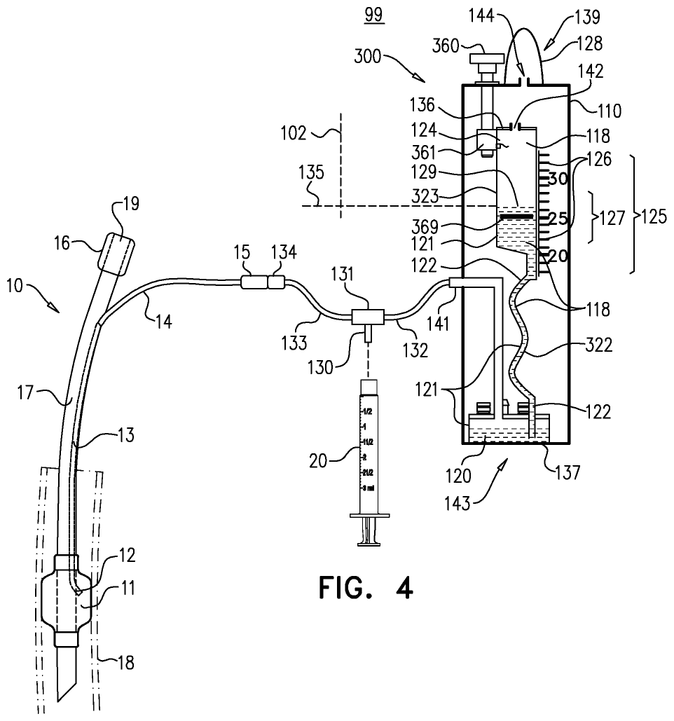

Reference is now made to FIG. 4, which is a schematic illustration of a cuff pressure stabilizer 300 for use with tracheal ventilation tube 10, in accordance with an application of the present invention. Except as described below, cuff pressure stabilizer 300 is generally similar to cuff pressure stabilizer 100, described hereinabove with reference to FIG. 1, and may implement any of the features thereof, mutatis mutandis.

Liquid column container 118 is shaped so as to define a wider portion 323 and a narrower portion 322 axially between wider portion 323 and fluid reservoir 120. Wider portion 323 has an average cross-sectional area, measured in (a) horizontal plane 135, described hereinabove with reference to FIG. 1, and/or (b) a plane perpendicular to a longitudinal axis of liquid column container 118. Narrower portion 322 has an average cross-sectional area, measured in horizontal plane 135 and/or the above-mentioned plane. For example, the average cross-sectional area of wider portion 323 may equal at least 200% of the average cross-sectional area of narrower portion 322, such as at least 300%, at least 400%, or at least 500%.

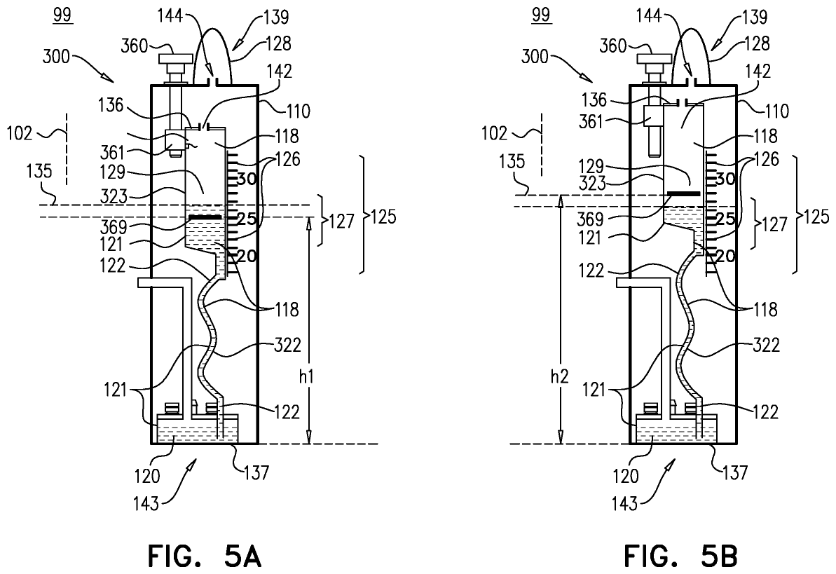

Typically, all axial locations along liquid column container 118 corresponding to the pressure of the gas in fluid reservoir 120 at the respective plurality of pressures in relevant pressure range 127 fall within wider portion 323. However, this may not be the case if a healthcare worker axially adjusts wider portion 323 beyond normal clinical limits (in configurations in which cuff pressure stabilizer 300 is arranged to provide an adjustable distance between wider portion 323 and fluid reservoir 120, such as described hereinbelow with reference to FIGS. 5A-B). Typically, all axial locations along liquid column container 118 corresponding to a pressure of the gas in fluid reservoir 120 of between 5 and 15 cm H2O fall within narrower portion 322.

For some applications, wider portion 323 has a length of:
at least 2 cm, such as at least 3 cm, and/or no more than 10 cm, such as no more than 5 cm,
the quotient of (a) at least 6 cm, such as at least 9 cm, and/or no more than 30 cm, such as no more than 15 cm, divided by (b) the specific gravity of liquid 121 with reference to water at 4 degrees Celsius at 1 atm, and/or
the quotient of (a) at least 6 cm, such as at least 9 cm, and/or no more than 30 cm, such as no more than 15 cm, divided by (b) the specific gravity of liquid 121 with reference to water at 20 degrees Celsius at 1 atm.

For some applications, when liquid column container 118 is oriented in the aligned orientation, wider portion 323 has an inner cross-sectional area, measured in horizontal plane 135 and/or the above-mentioned plane, at a plurality of (such as at most or all) axial locations along wider portion 323, of (a) at least 0.16 cm2 (e.g., at least 0.25 cm2, 0.5 cm2, or 1 cm2), (b) at least the product of 0.16 cm2 (e.g., at least 0.25 cm2, 0.5 cm2, or 1 cm2) and a specific gravity of liquid 121 with reference to water at 4 degrees Celsius at 1 atm, (c) at least the product of 0.16 cm2 (e.g., at least 0.25 cm2, 0.5 cm2, or 1 cm2) and a specific gravity of liquid 121 with reference to water at 20 degrees Celsius at 1 atm, and/or (d) less than 2 cm2. For some applications, when liquid column container 118 is oriented in the aligned orientation, wider portion 323 has an average inner cross-sectional area, measured in horizontal plane 135 and/or the above-mentioned plane, of (a) at least 0.16 cm2 (e.g., at least 0.25 cm2, 0.5 cm2, or 1 cm2), (b) at least the product of 0.16 cm2 (e.g., at least 0.25 cm2, 0.5 cm2, or 1 cm2) and a specific gravity of liquid 121 with reference to water at 4 degrees Celsius at 1 atm, (c) at least the product of 0.16 cm2 (e.g., at least 0.25 cm2, 0.5 cm2, or 1 cm2) and a specific gravity of liquid 121 with reference to water at 20 degrees Celsius at 1 atm, and/or (d) less than 2 cm2. For some applications, when liquid column container 118 is oriented in the aligned orientation, narrower portion 322 has an inner cross-sectional area, measured in horizontal plane 135 and/or the above-mentioned plane, of less than 0.16 cm2, such as less than 0.09 cm2, at most or all axial locations along narrower portion 322.

Reference is now made to FIGS. 5A and 5B, which are schematic illustrations of cuff pressure stabilizer 300 adjusted in respective different states, in accordance with an application of the present invention. For some applications, cuff pressure stabilizer 300 is arranged to provide an adjustable distance between wider portion 323 of liquid column container 118 and fluid reservoir 120. For example, cuff pressure stabilizer 300 is shown in FIG. 2B with wider portion 323 at a greater distance from fluid reservoir 120 than in FIG. 2A. Providing the adjustable difference allows a healthcare worker to optimize cuff pressure stabilizer 300 for a desired target pressure and target pressure range, while not requiring additional liquid 121. For example, some patients that are ventilated at higher pressure need higher average cuff pressures, e.g., 28 cm H2O or 30 cm H2O, rather than the typical 25 cm H2O target. There is also a range of preferred target pressures even for patients ventilated at normal pressure, depending on the patient's particular circumstances and the medical opinion of the physician.

For some applications, cuff pressure stabilizer 300 is arranged such that the adjustable distance can vary by at least 1 cm. Alternatively or additionally, for some applications, cuff pressure stabilizer 300 is arranged such that the adjustable distance can vary by (a) at least the quotient of (i) 3 cm divided by (ii) the specific gravity of liquid 121 with reference to water at 4 degrees Celsius at 1 atm, and/or (b) at least the quotient of (i) 3 cm divided by (ii) the specific gravity of liquid 121 with reference to water at 20 degrees Celsius at 1 atm.

For some applications, cuff pressure stabilizer 300 comprises a mechanical user control element 360, which is arranged to set the adjustable distance, for example by rotation, e.g., with respect to a threaded connector 361 that is fixed to wider portion 363. Alternatively, mechanical user control element 360 may axially slide with respect to casing 110 upon application of a sufficient force to overcome friction preventing such sliding.

For some applications, such as shown in FIGS. 4 and 5A-B, cuff pressure stabilizer 300 is arranged to provide the adjustable distance by providing an adjustable axial position of wider portion 323 with respect to casing 110. For example, wider portion 323 of liquid column container 118 may be axially-slidably coupled to casing 110. Typically, for these applications, fluid reservoir 120 is fixed to casing 110, and/or casing 110 has pressure indicia markings 126. Cuff pressure stabilizer 300 may be arranged to limit the axial endpoints of the adjustable axial position of wider portion 323. For example, casing 110 may comprise one or more stoppers that limit the adjustable axial position of wider portion 323, and/or mechanical user control element 360 may be configured to provide a limited range of adjustable axial positions.

For other application (configuration not shown), fluid reservoir 120 is axially-slidably coupled to casing 110. Typically, for these applications, wider portion 323 of liquid column container 118 is fixed to casing 110.

For some applications, wider portion 323 of liquid column container 118 has a target-pressure indicator marker 369 (e.g., a horizontal line), which is axially slidable with respect to pressure indicia markings 126, which, as mentioned above, are typically provided on casing 110. The healthcare worker may set target-pressure indicator marker 369 to indicate a desired target pressure in inflatable cuff 11, and then inflate inflatable cuff 11 at least approximately to this target pressure. Such setting of target-pressure indicator marker 369, by axially moving target-pressure indicator marker 369 with respect to pressure indicia markings 126, has the effect of adjusting the adjustable distance between wider portion 323 of liquid column container 118 and fluid reservoir 120.

Target-pressure indicator marker 369 is disposed on wider portion 323 so as to delineate (a) an upper portion of wider portion 323 above target-pressure indicator marker 369 and (b) a lower portion of wider portion 323 below target-pressure indicator marker 369. For some applications, the volume of the upper portion equals:
at least 100% of the volume of the lower portion, such as at least 150%, e.g., between 150% and 250%, such as 200% of the volume of the lower portion,
between 1 and 2.5 cc, e.g., 2 cc,
the quotient of (a) between 3 and 10 cc, e.g., between 4 and 8 cc divided by (b) the specific gravity of liquid 121 with reference to water at 4 degrees Celsius at 1 atm, and/or
the quotient of (a) between 3 and 10 cc, e.g., between 4 and 8 cc divided by (b) the specific gravity of liquid 121 with reference to water at 20 degrees Celsius at 1 atm.
   For some applications, the volume of the lower portion equals:
between 0.75 and 1.25 cc, e.g., 1 cc,
the quotient of (a) between 1.5 and 5 cc, e.g., between 2 and 4 cc divided by (b) the specific gravity of liquid 121 with reference to water at 4 degrees Celsius at 1 atm, and/or
the quotient of (a) between 1.5 and 5 cc, e.g., between 2 and 4 cc divided by (b) the specific gravity of liquid 121 with reference to water at 20 degrees Celsius at 1 atm.

For some applications, at least an axial portion of narrower portion 322 of liquid column container 118 is flexible, so as to provide a variable axial length to narrower portion 322. For some of these applications, the at least an axial portion of narrower portion 322 of liquid column container 118 is elastic. For other applications, at least an axial portion of narrower portion 322 of liquid column container 118 is telescopically adjustable, so as to provide a variable axial length to narrower portion 322.

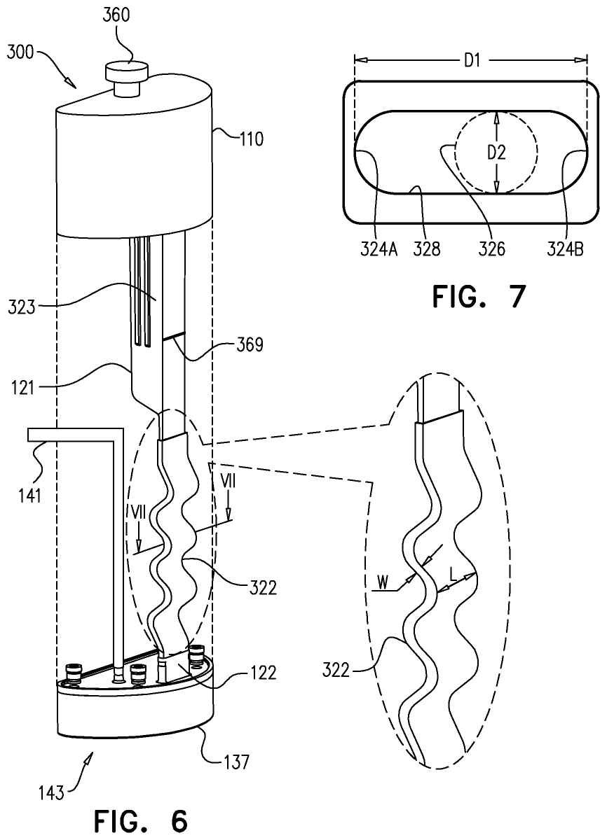

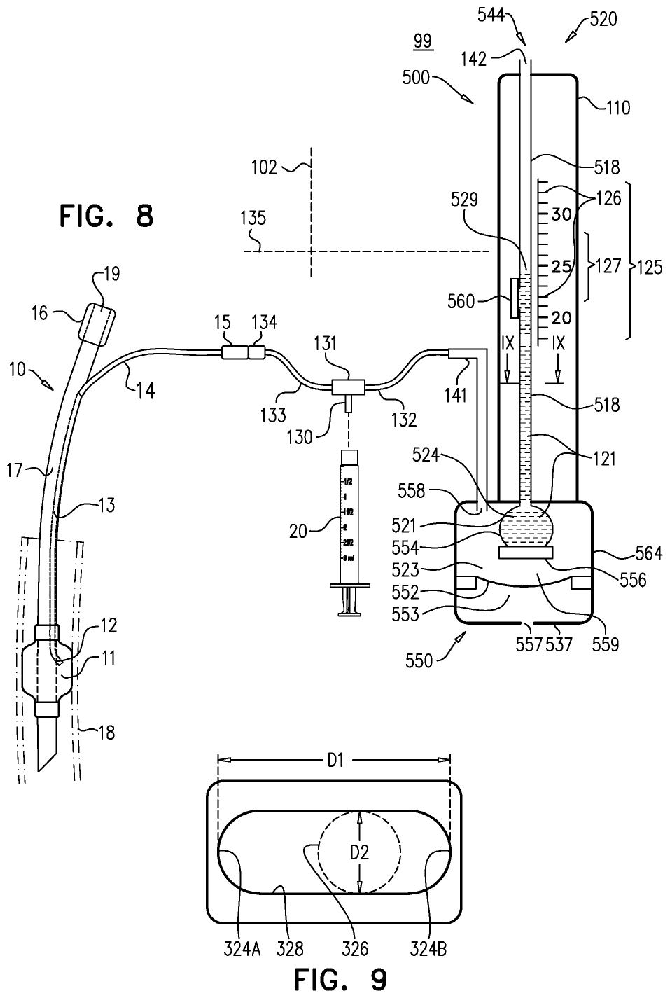

Reference is still made to FIGS. 4 and 5A-B, and is additionally made to FIG. 6, which is another schematic illustration of cuff pressure stabilizer 300, and to FIG. 7, which is a cross-sectional view taken along the line VII-VII of FIG. 6, in accordance with an application of the present invention. For some applications, narrower portion 322 of liquid column container 118 is generally thin and flat.

For some applications, at each of all axial locations along narrower portion 322 of liquid column container 118, liquid column container 118:
has a largest inner dimension D₁ equal to a greatest distance between any two points 324A and 324B within liquid column container 118 in (a) horizontal plane 135, described hereinabove with reference to FIG. 1, and/or (b) a plane perpendicular to the longitudinal axis of liquid column container 118, and
can encompass a largest circle 326 in the horizontal plane.
   At most or all (e.g., all) of the axial locations along narrower portion 322 of liquid column container 118, a ratio of (a) the largest inner dimension D₁ to (b) the diameter D₂ of circle 326 equals at least 2:1, such as at least 4:1, e.g., at least 8:1. It is to be understood that circle 326 is not an element of cuff pressure stabilizer 300, but rather a geometric construct used to describe a structural property of cuff pressure stabilizer 300.

For some applications, the largest inner dimension D₁ equals at least 4 mm at most or all of the axial locations along narrower portion 322 of liquid column container 118, such as at least 6 mm, e.g., at least 8 mm Alternatively or additionally, for some applications, the diameter D₂ of circle 326 is no more than 4 mm at most or all of the axial locations along narrower portion 322 of liquid column container 118, such as no more than 2 mm, e g , no more than 1 mm, such as no more than 0.5 mm

For some applications, at most or all of the axial locations along narrower portion 322 of liquid column container 118, liquid column container 118 has a non-circular cross-sectional shape, such as a rectangle, an oblong shape, an ellipse, or a crescent. For applications in which the cross-sectional shape is a rectangle, a length L of the rectangular typically equals at least 200% of a width W of the rectangle, such as at least 300% or at least 400%. For applications in which the cross-sectional shape is an ellipse, a length of the major axis of the ellipse typically equals at least 200% of a length of the minor axis of the ellipse, such as at least 300% or at least 400%.

Alternatively or additionally, for some applications, at each of the axial locations along narrower portion 322 of liquid column container 118, at least 80% of the inner cross-sectional area is within 1 mm of an inner surface 328 of liquid column container 118, such as within 0.75 mm, e.g., within 0.5 mm, such as within 0.2 mm, of inner surface 328. For some applications, at each of the axial locations along narrower portion 322 of liquid column container 118, less than 10% of the inner cross-sectional area is within 0.1 mm, such as within 0.2 mm, of inner surface 328 of liquid column container 118. For some applications, the inner cross-sectional area is less than 0.09 cm2 at most or all axial locations along narrower portion 322 of liquid column container 118, when liquid column container 118 is oriented in the aligned orientation.

Reference is made to FIGS. 4-7. The generally thin flat shape of the cross-sectional shape of narrower portion 322 of liquid column container 118 prevents gas bubbles from occluding narrower portion 322. The surface tension of the bubble causes the bubble not to reach the edges of the liquid column container. By contrast, in configurations in which narrower portion 322 is circular in cross-section, such as described hereinabove with reference to FIGS. 1-3B, gas bubbles may sometimes occlude the narrower portion, particularly if the diameter of the tube is very small. The generally thin flat shape of the cross-sectional shape generally prevents such occlusion. As a consequence, even if cuff pressure stabilizer 300 does not comprise orientation-sensitive valve assembly 150 or 250, described hereinabove with reference to FIGS. 1 and 2-3A, respectively, and gas bubbles enter liquid column container 118 upon excessive tilting of cuff pressure stabilizer 300, narrower portion 322 still does not become occluded.

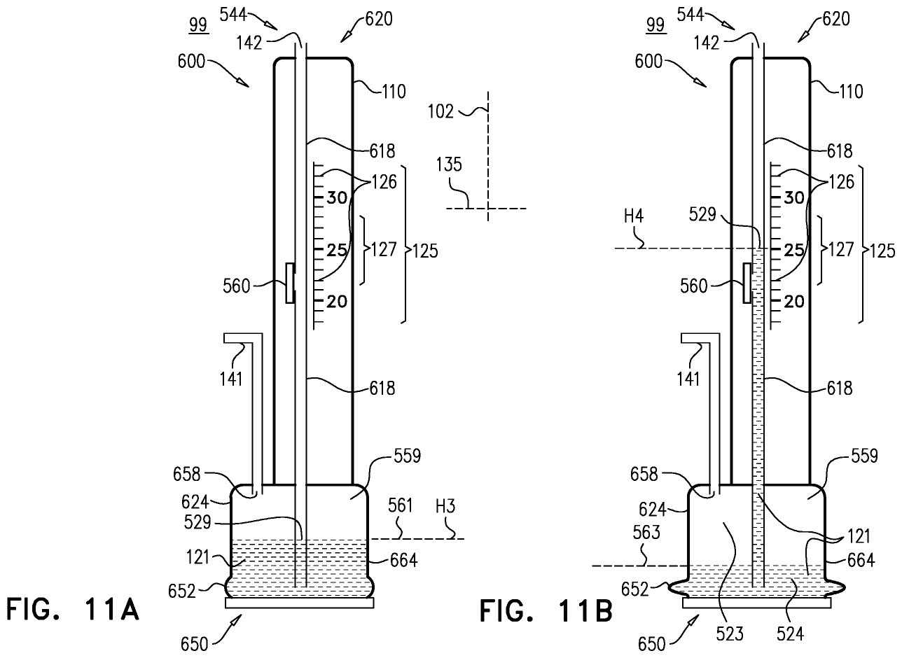

Reference is now made to FIG. 8, which is a schematic illustration of a cuff pressure stabilizer 500 for use with tracheal ventilation tube 10, in accordance with an application of the present invention, and to FIG. 9, which is a cross-sectional view taken along the line IX-IX of FIG. 8, in accordance with an application of the present invention. Except as described below, cuff pressure stabilizer 500 is generally similar to cuff pressure stabilizer 100, described hereinabove with reference to FIG. 1, and may implement any of the features thereof, mutatis mutandis.

Cuff pressure stabilizer 500 comprises a fluid reservoir 524 and a liquid column container 518. Liquid column container 518 is (a) open to atmosphere 99 at at least one site along liquid column container 518, (b) in fluid communication with fluid reservoir 524, and (c) in communication with the inflation lumen proximal port connector 134 via fluid reservoir 524. Cuff pressure stabilizer 500 comprises a buffer module 550, which is configured to provide automatic pressure regulation of inflatable cuff 11, while simultaneously continuously displaying the pressure in inflatable cuff 11. Cuff pressure stabilizer 500 further comprises an indicator module 520, which continuously displays the pressure in inflatable cuff 11.

Cuff pressure stabilizer 500 further comprises liquid 121, which is contained (a) in fluid reservoir 524, (b) in liquid column container 518, or (c) partially in fluid reservoir 524 and partially in liquid column container 518. Like cuff pressure stabilizer 100, cuff pressure stabilizer 500 has a plurality of pressure indicia markings 126 distributed along liquid column container 518 for measuring a height of liquid 121 in liquid column container 518. Typically, pressure indicia markings 126 are distributed evenly throughout at least relevant pressure range 127 of 23-27 cm H2O.

Typically, liquid 121 has (a) a density of between 1.5 and 5 g/cm3 at 4 degrees Celsius at 1 atm, such as less than 3.5 g at 4 degrees Celsius at 1 atm, e.g., less than 3 g at 4 degrees Celsius at 1 atm, e.g., between 1.5 and 3.5 g/cm3, such as between 1.5 and 3 g/cm3, and/or (b) a density of between 1.5 and 5 g/cm3 at 20 degrees Celsius at 1 atm, such as less than 3.5 g at 20 degrees Celsius at 1 atm, e.g., less than 3 g at 20 degrees Celsius at 1 atm, e.g., between 1.5 and 3.5 g/cm3, such as between 1.5 and 3 g/cm3. Such a density provides high resolution pressure readings in the above-mentioned relevant pressure range 127 of 23-27 cm H2O, without requiring liquid column container 518 to be very long and unwieldy, such as if liquid 121 was H2O.

For some applications, liquid 121 has (a) a viscosity of no more than 25 times a viscosity of water at 4 degrees Celsius at 1 atm, such as no more than 15, no more than 10, or no more than 5 times the viscosity of water at 4 degrees Celsius at 1 atm, and/or (b) a viscosity of no more than 25 times a viscosity of water at 20 degrees Celsius at 1 atm, such as no more than 15, no more than 10, or no more than 5 times the viscosity of water at 20 degrees Celsius at 1 atm. For some applications, liquid 121 comprises a solution of crystals (solute) dissolved in a liquid solvent (typically water), having a mass percent of between 65% and 85% (e.g., between 75% and 85%) (i.e., the mass of the liquid solvent (typically water) is only between 15% and 35% of the total mass of the solution). The dilution is selected based on the desired density of the liquid.

Typically, liquid 121 is non-toxic. As used in the present application, including in the claims, “non-toxic” has the meaning generally understood in the medical arts, i.e., that the full quantity of liquid 121 of the cuff pressure stabilizer, even if it is swallowed by the patient or comes in contact with the patient's skin, will not produce personal injury or illness to the patient. (The liquid is still “non-toxic” if it causes mild irritations upon coming contact in with the eyes (which is not an intended use of the liquid).) For example, criteria for ascertaining whether a substance is “toxic” are provided in the U.S. Federal Hazardous Substances Act (FHSA) and the Chemicals Act of Germany (Chemikaliengesetz—ChemG), as amended in 2008.

Typically, liquid 121 is non-flammable. Typically, liquid 121 is odorless. For some applications, liquid 121 comprises a tungstate-based liquid, e.g., selected from the group consisting of sodium polytungstate, sodium metatungstate, lithium polytungstate, and lithium metatungstate. For some applications, liquid 121 comprises at least two liquids, at least one of which has the density of between 1.5 and 5 g/cm3 at 4 degrees Celsius at 1 atm, and at least one of which has a density of less than 1.5 g/cm3 at 4 degrees Celsius at 1 atm. For some applications, liquid 121 comprises at least two liquids, at least one of which has the density of between 1.5 and 5 g/cm3 at 20 degrees Celsius at 1 atm, and at least one of which has a density of less than 1.5 g/cm3 at 20 degrees Celsius at 1 atm.

For some applications, a volume of liquid 121 is at least 0.5 cc, no more than 4 cc, and/or between 0.5 and 4 cc, such as at least 1 cc, no more than 2 cc, and/or between 1 and 2 cc.

For some applications, liquid column container 518 is in pressure communication with inflation lumen proximal port connector 134 via fluid reservoir 524. For some of these applications, fluid reservoir 524 comprises at least one wall 521 that comprises a pressure-communicating movable wall 554, and liquid column container 518 is in pressure communication with inflation lumen proximal port connector 134 via pressure-communicating movable wall 554 of fluid reservoir 524. For some applications (as shown), pressure-communicating movable wall 554 comprises a flexible membrane, which typically is elastic or pliable, while for other applications (not shown), pressure-communicating movable wall 554 comprises another movable structure, such as bellows.

For some applications, cuff pressure stabilizer 500 comprises a gas container 523, which (a) extends to inflation lumen proximal port connector 134, (b) contains some of the gas, (c) is not in liquid communication with fluid reservoir 524, and (d) comprises at least one wall that comprises a volume-compensation movable wall 552, which is in pressure communication with atmosphere 99 on the other side of the movable wall. For some applications (as shown), volume-compensation movable wall 552 comprises a flexible membrane, which typically is elastic or pliable, while for other applications (not shown), volume-compensation movable wall 552 comprises another movable structure, such as bellows. When inflatable cuff 11 is squeezed and its volume therefore decreases, gas is pushed from the inflatable cuff into gas container 523. As a result, volume-compensation movable wall 552 moves (e.g., stretches, for applications in which the movable wall comprises a flexible membrane) and thereby increases the total volume of gas container 523 to accommodate the addition gas. In addition, a liquid upper surface 529 of liquid 121 in liquid column container 518 moves upward, also increasing the volume of gas container 523.

For some of these applications, fluid reservoir 524 comprises the at least one wall 521 that comprises pressure-communicating movable wall 554, and liquid column container 518 is in pressure communication with gas container 523 via pressure-communicating movable wall 554 of fluid reservoir 524. Pressure-communicating movable wall 554 thus prevents fluid communication between fluid reservoir 524 and gas container 523, while allowing pressure communication therebetween. Typically, pressure-communicating movable wall 554 is disposed at least partially within gas container 523.

Pressure-communicating movable wall 554 is typically deformable (e.g., elastic and/or pliable). As a result, when the gas pressure increases in gas container 523, the gas presses and moves pressure-communicating movable wall 554, thereby pushing liquid 121 in fluid reservoir 524 upward within liquid column container 518, and thus also elevating liquid upper surface 529 of liquid 121 in liquid column container 518. For some applications, pressure-communicating movable wall 554 is supported at its bottom by a rigid platform 556, which maintains the lowest point of liquid 121 at a fixed reference height.

For some applications, gas container 523 comprises a buffer chamber 564, which is shaped so as to define a chamber inlet port 558 that is in fluid communication with inflation lumen proximal port connector 134, such as via gas inlet 141. For some applications, buffer chamber 564 has a volume of at least 1 cc when a pressure of the gas in buffer chamber 564 is 25 cm H2O. For some applications, a volume of buffer chamber 564 increases by at least 1 cc, such as by at least 2 cc, and/or by no more than 5 cc, when a pressure of gas 559 in buffer chamber 564 increases from 25 cm H2O to 30 cm H2O. For some applications, at least one wall of buffer chamber 564 comprises volume-compensation movable wall 552, described above.

Buffer chamber 564 typically comprises a buffer chamber casing 537. For some applications, pressure-communicating movable wall 554 is disposed at least partially within buffer chamber 564, e.g., within buffer chamber casing 537. For some of these applications, buffer chamber casing 537 is shaped so as to define, in addition to buffer chamber 564, an enclosed volume-compensation movable wall expansion space 553, into which volume-compensation movable wall 552 can expand. For such applications, buffer chamber casing 537 typically is shaped so as to define a buffer chamber air environment port 557 between enclosed volume-compensation movable wall expansion space 553 and atmosphere 99.

For some applications, liquid column container 518 comprises a dissolvable wall portion 560 that is dissolvable in water. Typically, dissolvable wall portion 560 defines a perforation therethrough that is configured to become permeable to liquid 121 through the perforation after total time of at least 3 days, e.g., at least 7 days, at least 10 days, or at least 14 days, and/or less than 30 days of contact with liquid 121. Typically, dissolvable wall portion 560 remains impermeable to liquid 121 for at least 48 hours, e.g., at least 1 week, at least 2 weeks, or at least 3 weeks, of contact between liquid 121 and dissolvable wall portion 560. For some applications, before the first use of cuff pressure stabilizer 500, an elongate plug is disposed in liquid column container 518 (typically through opening 142) and reaches below the bottom end of dissolvable wall portion 560, thereby preventing liquid 121 from coming into contact with dissolvable wall portion 560 until the plug is removed. Dissolvable wall portion 560 may preserve sterility of cuff pressure stabilizer 500 by preventing reuse of the stabilizer for more than one patient.

For some applications, when liquid column container 518 is oriented in an aligned orientation in which pressure indicia markings 126 reflect, to within 1 cm H2O, pressure of the gas in buffer chamber 564 at least in relevant pressure range 127 of 23-27 cm H2O: dissolvable wall portion 560 is disposed at least partially below an axial location along liquid column container 518 corresponding to a pressure of the gas in buffer chamber 564 of 23 cm H2O. For some applications, when liquid column container 518 is oriented in the aligned orientation and the pressure of the gas in buffer chamber 564 equals ambient air pressure, dissolvable wall portion 560 is disposed above liquid upper surface 529 of liquid 121 in liquid column container 518.

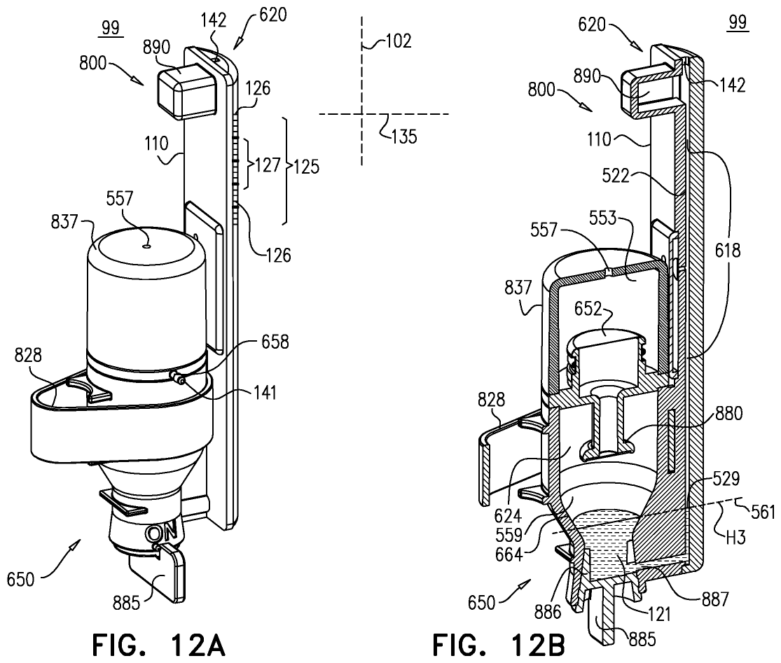

Reference is now made to FIGS. 10A-B, which are schematic illustrations of cuff pressure stabilizer 500 in resting and pressurized states, respectively, in accordance with an application of the present invention. In the resting state illustrated in FIG. 10A, the pressure of gas 559 in buffer chamber 564 is equal to the ambient air pressure of atmosphere 99. In this initial resting configuration, liquid upper surface 529 of liquid 121 in liquid column container 518 is at a height H1.

As illustrated in FIG. 10B, when the buffer gas pressure increases in gas container 523, pressure-communicating movable wall 554 is pressed and moves so as to push liquid 121 upward within liquid column container 518, thereby elevating liquid upper surface 529 to height H2, higher than height H1. In addition, when the buffer gas pressure increases in gas container 523, volume-compensation movable wall 552 moves (e.g., stretches, for applications in which the movable wall comprises a flexible membrane), as illustrated in FIG. 10B.

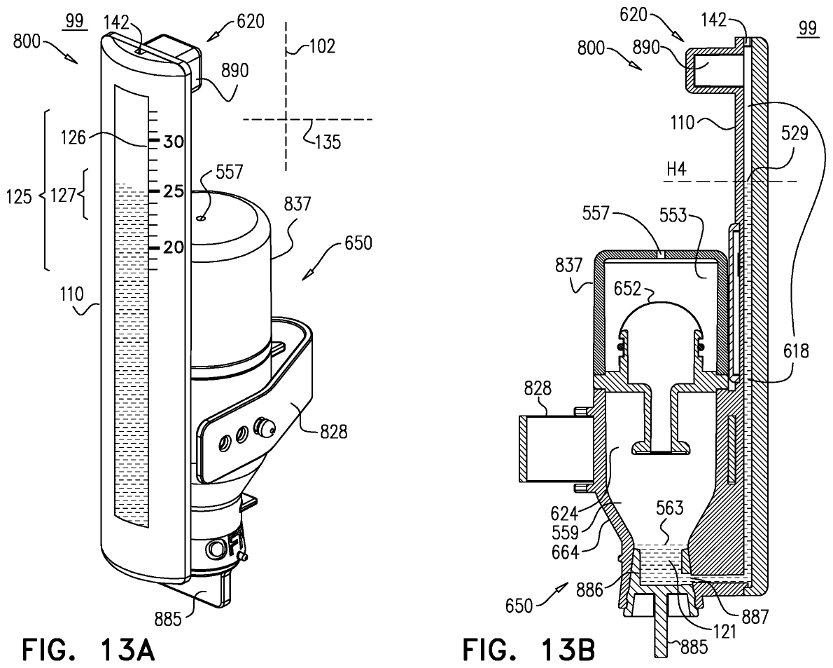

Reference is now made to FIGS. 11A-B, which are schematic illustrations of a cuff pressure stabilizer 600 in resting and pressurized states, respectively, for use with tracheal ventilation tube 10, in accordance with an application of the present invention. Except as described below, cuff pressure stabilizer 600 is generally similar to cuff pressure stabilizer 100, described hereinabove with reference to FIG. 1, and cuff pressure stabilizer 500, described hereinabove with reference to FIGS. 8-10B, and may implement any of the features of either of these pressure stabilizers, mutatis mutandis.

Cuff pressure stabilizer 600 comprises a fluid reservoir 624, and a liquid column container 618, which is (a) open to atmosphere 99 at at least one site along liquid column container 618, (b) in fluid communication with fluid reservoir 624, and (c) in communication with the inflation lumen proximal port connector 134 via fluid reservoir 624. Cuff pressure stabilizer 600 comprises a buffer module 650, which is configured to provide automatic pressure regulation of inflatable cuff 11, while simultaneously continuously displaying the pressure in inflatable cuff 11. Cuff pressure stabilizer 600 further comprises an indicator module 620, which continuously displays the pressure in inflatable cuff 11.

Typically, fluid reservoir 624 contains some of gas 559, and liquid column container 618 is in fluid communication with inflation lumen proximal port connector 134 via fluid reservoir 624. Consequently, gravity causes gas 559 in fluid reservoir 624 to be above the portion of liquid 121 in fluid reservoir 624.

For some applications, fluid reservoir 624 extends to inflation lumen proximal port connector 134, and comprises at least one wall 630 that comprises a volume-compensation movable wall 652, which is in pressure communication with atmosphere 99. For some applications, fluid reservoir 624 comprises a buffer chamber 664, which is shaped so as to define a chamber inlet port 658 that is in fluid communication with inflation lumen proximal port connector 134, such as via gas inlet 141. For some of these applications, buffer chamber 664 comprises the at least one wall 630 that comprises volume-compensation movable wall 652. For some applications, buffer chamber 664 has a volume of at least 2 cc when gas 559 in buffer chamber 664 is at a pressure of 25 cm H2O.

In the resting state illustrated in FIG. 11A, the pressure of gas 559 in buffer chamber 664 is equal to the ambient air pressure of atmosphere 99. In this initial resting configuration, both the liquid upper surface 529 of liquid 121 in liquid column container 618 and the height 561 of the liquid surface in buffer chamber 664 are at a height H3.

As illustrated in FIG. 11B, increase in the buffer gas pressure in buffer chamber 664 pushes liquid 121 partially from fluid reservoir 624 and upward within liquid column container 618, thereby elevating liquid upper surface 529 within liquid column container 618 to a height H4, higher than height H3, and the height 563 of the liquid surface in buffer chamber 664 is lower than height H3. For some applications, a volume of buffer chamber 664 increases by at least 1 cc when a pressure of gas 559 in buffer chamber 664 increases from 25 cm H2O to 30 cm H2O. Alternatively or additionally, for some applications, a volume of buffer chamber 664 increases by at least 1 cc and/or less than 3 cc when a pressure of gas 559 in buffer chamber 664 increases from 25 cm H2O to 28 cm H2O. In addition, as illustrated in FIG. 11B, the increase of buffer fluid gas pressure induces pressure on volume-compensation movable wall 652 and deforms it to a stretched-out configuration, thereby increasing the total volume of buffer chamber 664.

For some applications, fluid reservoir 624 contains some of gas 559, and when liquid column container 618 is oriented in an aligned orientation in which pressure indicia markings 126 reflect, to within 1 cm H2O, pressure of the gas in fluid reservoir 624 at least in relevant pressure range 127 of 23-27 cm H2O: dissolvable wall portion 560 is disposed at least partially below an axial location along liquid column container 618 corresponding to a pressure of the gas in fluid reservoir 624 of 23 cm H2O. For some applications, when liquid column container 618 is oriented in an aligned orientation and the pressure of gas 559 in fluid reservoir 624 equals ambient air pressure, dissolvable wall portion 560 is disposed above liquid upper surface 529 of liquid 121 in liquid column container 618.

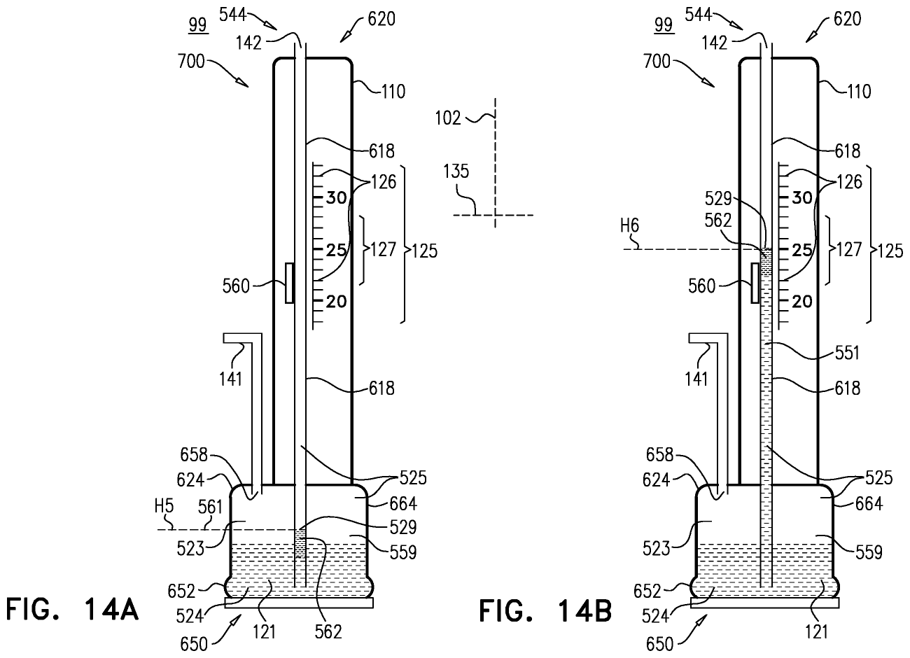

Reference is now made to FIGS. 12A-B and 13A-B, which are schematic illustrations of a cuff pressure stabilizer 800 in resting and pressurized states, respectively, for use with tracheal ventilation tube 10, in accordance with an application of the present invention. FIGS. 12B and 13B are cross-sectional views of FIGS. 12A and 13A, respectively. Except as described below, cuff pressure stabilizer 800 is generally similar to cuff pressure stabilizer 600, described hereinabove with reference to FIGS. 11A-B, and may implement any of the features of cuff pressure stabilizer 600, mutatis mutandis.

For some applications, buffer chamber 664 comprises a buffer chamber casing 837. For some applications, volume-compensation movable wall 652 is disposed at least partially within buffer chamber 664, e.g., within buffer chamber casing 837. Buffer chamber casing 837 typically is shaped so as to define buffer chamber air environment port 557 between enclosed volume-compensation movable wall expansion space 553 and atmosphere 99.

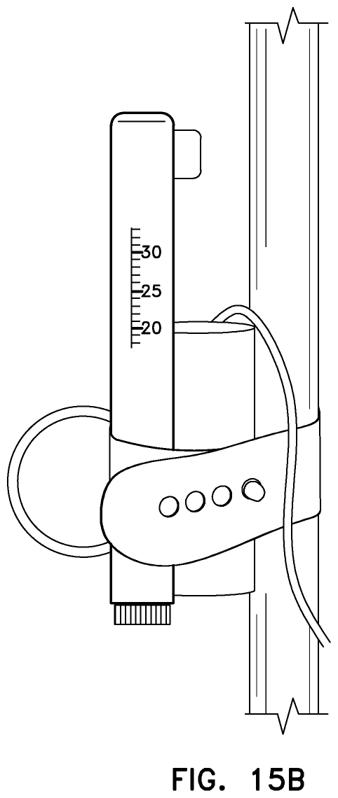

Typically, cuff pressure stabilizer 800 comprises a coupling element 828, which may comprise, for example, a strap (as shown), a gripper (not shown), or a clamp (not shown), which is coupleable to a vertical pole (e.g., a vertical IV pole), such as shown for the prototypes in FIGS. 15A-B, described hereinbelow. Alternatively, cuff pressure stabilizer 800 may comprise a hook or loop, such as described hereinabove with reference to FIG. 1. The other cuff pressure stabilizers described herein may also comprise coupling element 828 or a hook or loop.

For some applications, buffer chamber 664 is shaped so as to define an internal spill-prevention element 880, which may be shaped as an inverted bottle neck. Spill-prevention element 880 creates a pool such that if the device is laid on its side or even turned upside down, liquid 121 will not spill by gravity through buffer chamber air environment port 557.

For some applications, cuff pressure stabilizer 800 comprises an on/off valve 885, which enables/disables fluid communication between buffer chamber 664 and liquid column container 618. In the illustrated configuration, valve 885 is switched on/off by rotation around the vertical axis, e.g., upon turning by 180 degrees, a fluid passage 887 to liquid column container 618 is turned away and instead a wall 886 seals the fluid communication between buffer chamber 664 and liquid column container 618.

For some applications, cuff pressure stabilizer 800 comprises a pressure-release chamber 890. Pressure-release chamber 890 is typically sized to have a volume larger than that of the full liquid content. If the pressure rises to a level above that of a level of the bottom of pressure-release chamber 890, liquid 121 is collected within pressure-release chamber 890. Liquid 121 is the only gas-seal that prevents escape of gas from the cuff to atmosphere 99. Therefore, if the cuff pressure rises to a pressure substantially above the level of the bottom of pressure-release chamber 890, all of liquid 121 is collected in pressure-release chamber 890, and gas escapes from the cuff to atmosphere 99, thereby releasing the excess pressure. The level of liquid 121 then falls again to recreate the gas seal so as to prevent further gas leakage from the cuff to atmosphere 99. Altogether, pressure-release chamber 890 operates as an effective pressure-release valve in which the pressure limit is set by the height of pressure-release chamber 890.

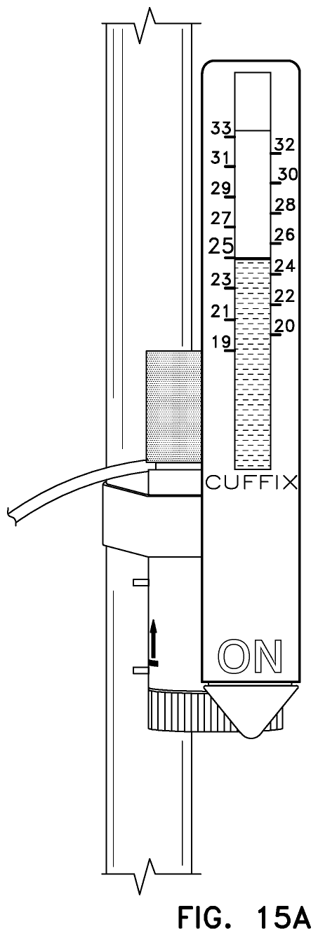

Reference is now made to FIGS. 14A-B, which are schematic illustrations of another configuration of cuff pressure stabilizer 600 in resting and pressurized states, respectively, for use with tracheal ventilation tube 10, in accordance with an application of the present invention. This configuration is similar to the configuration described hereinabove with reference to FIGS. 11A-B, with the additional feature that liquid 121 comprises two distinct liquids: a heavier liquid, which typically has of a density of between 1.3 and 3.3 g/cm3 on average, and a lighter liquid 562. As a result, liquid upper surface 529 of the total buffer liquid is at the boundary of lighter liquid 562 with ambient air. This is not meant to be limiting, in the sense that the extension of such configuration to use of more than two liquids of different densities (e.g., 3 or more) is straightforward. The features of this configuration may alternatively implemented in combination with any of the other cuff pressure stabilizers described herein.

Reference is now made to FIGS. 15A-B, which are photographs of prototypes of the cuff pressure stabilizer of FIGS. 12A-B and 13A-B, in accordance with respective applications of the present invention. As can be seen, each of the prototype cuff pressure stabilizers is coupled to a vertical IV pole.

Although cuff pressure stabilizers 100, 200, 300, 500, 600, and 800 have been described as being used with inflatable cuff 11 of tracheal ventilation tube 10, cuff pressure stabilizers 100, 200, 300, 500, 600, and 800 may alternatively be used with other inflatable chambers of other medical devices or non-medical devices. For example, the inflatable chamber may be a Foley catheter balloon, a gastric balloon, a balloon of colonoscope, or a balloon of an endoscope.

In the description and claims of the present application, each of the verbs, “comprise,” “include” and “have,” and conjugates thereof, are used to indicate that the object or objects of the verb are not necessarily a complete listing of members, components, elements or parts of the subject or subjects of the verb. The articles “a” and “an” are used herein to refer to one or to more than one (i.e., to at least one) of the grammatical object of the article. By way of example, “an element” means one element or more than one element. The term “including” is used herein to mean, and is used interchangeably with, the phrase “including but not limited to.” The term “or” is used herein to mean, and is used interchangeably with, the term “and/or,” unless context clearly indicates otherwise. The term “such as” is used herein to mean, and is used interchangeably, with the phrase “such as but not limited to.”

All publications, patent applications, patents, and other references mentioned herein are incorporated by reference in their entirety. In case of conflict, the present patent specification, including definitions, will prevail. In addition, the materials, methods, and examples are illustrative only and not intended to be limiting.

For brevity, some explicit combinations of various features are not explicitly illustrated in the figures and/or described. It is now disclosed that any combination of the method or device features disclosed herein can be combined in any manner—including any combination of features—any combination of features can be included in any embodiment and/or omitted from any embodiments.

As used in the present application, including in the claims, a “fluid” comprises liquid and/or gas.

Although applications of the present invention have generally been described as for use with tracheal ventilation tube 10, they may also be used with other catheters, such as tracheostomy catheters.

The scope of the present invention includes embodiments described in the following applications, which are assigned to the assignee of the present application and are incorporated herein by reference. In an embodiment, techniques and apparatus described in one or more of the following applications are combined with techniques and apparatus described herein:

PCT Publication WO/2012/131626 to Einav et al.

GB 2482618 A to Einav et al.;

UK Application GB 1119794.4, filed Nov. 16, 2011;

U.S. Provisional Application 61/468,990, filed Mar. 29, 2011;

U.S. Provisional Application 61/473,790, filed Apr. 10, 2011;

U.S. Provisional Application 61/483,699, filed May 8, 2011;

U.S. Provisional Application 61/496,019, filed Jun. 12, 2011;

U.S. Provisional Application 61/527,658, filed Aug. 26, 2011;

U.S. Provisional Application 61/539,998, filed Sep. 28, 2011;

U.S. Provisional Application 61/560,385, filed Nov. 16, 2011;

U.S. Provisional Application 61/603,340, filed Feb. 26, 2012;

U.S. Provisional Application 61/603,344, filed Feb. 26, 2012;

U.S. Provisional Application 61/609,763, filed Mar. 12, 2012;

U.S. Provisional Application 61/613,408, filed Mar. 20, 2012;

U.S. Provisional Application 61/635,360, filed Apr. 19, 2012;

U.S. Provisional Application 61/655,801, filed Jun. 5, 2012;

U.S. Provisional Application 61/660,832, filed Jun. 18, 2012;

U.S. Provisional Application 61/673,744, filed Jul. 20, 2012;

PCT Publication WO 2013/030821 to Zachar et al.;

U.S. Pat. No. 8,999,074 to Zachar et al.;

U.S. Provisional Application 62/305,567, filed Mar. 9, 2016;

U.S. Provisional Application 62/402,024, filed Sep. 30, 2016; and

U.S. Provisional Application 62/405,115, filed Oct. 6, 2016

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-52. (canceled)

53. Apparatus for use in contact with the atmosphere of the Earth and for use with a gas and a catheter having an inflatable cuff, an inflation lumen, and an inflation lumen proximal port, the apparatus comprising a cuff pressure stabilizer, which is configured to provide automatic pressure regulation of the inflatable cuff of the catheter, and which comprises:
an inflation lumen proximal port connector, which is configured to form an air-tight seal with the inflation lumen proximal port of catheter, so as to assume a connected configuration;
a gas inlet, which is in fluid communication with the inflation lumen proximal port connector; and
a gas container, which (a) is in fluid communication with the inflation lumen proximal port connector via the gas inlet, (b) contains some of the gas, (c) comprises (i) at least one wall that comprises a volume-compensation movable wall, which is in pressure communication with the atmosphere on the other side of the volume-compensation movable wall, wherein the volume-compensation movable wall comprises a flexible elastic membrane, and (ii) a buffer chamber, which is shaped so as to define a chamber inlet port that is in fluid communication with the inflation lumen proximal port connector via the gas inlet,
wherein the cuff pressure stabilizer is configured such that when the inflation lumen proximal port connector is in the connected configuration, a combined air-flow resistance between an interior of the inflatable cuff and the gas container is such that a transient pressure difference of 5 cm H2O between the interior of inflatable cuff and the gas container results in gas flow from the interior of the inflatable cuff to the gas container at a slow rate of less than 0.1 cc per second that delays a response of the automatic pressure regulation of the cuff pressure stabilizer.

54. The apparatus according to claim 53, wherein at least one wall of the buffer chamber comprises the volume-compensation movable wall.

55. The apparatus according to claim 54, wherein the buffer chamber is configured such that a volume of the buffer chamber increases by at least 1 cc when a pressure of the gas in the buffer chamber increases from 25 cm H2O to 30 cm H2O.

56. The apparatus according to claim 55, wherein the buffer chamber is configured such that the volume of the buffer chamber increases by at least 2 cc when the pressure of the gas in the buffer chamber increases from 25 cm H2O to 30 cm H2O.

57. The apparatus according to claim 54, wherein the buffer chamber is configured such that a volume of buffer chamber increases by no more than 5 cc when a pressure of the gas in the buffer chamber increases from 25 cm H2O to 30 cm H2O.

58. The apparatus according to claim 53, wherein the cuff pressure stabilizer further comprises a buffer chamber casing shaped so as to define an enclosed volume-compensation movable wall expansion space into which the volume-compensation movable wall can expand.

59. The apparatus according to claim 58, wherein the buffer chamber casing is shaped so as to define a buffer chamber air environment port between the enclosed volume-compensation movable wall expansion space and the atmosphere.

60. The apparatus according to claim 53, wherein the cuff pressure stabilizer is configured such that when the inflation lumen proximal port connector is in the connected configuration, the transient pressure difference of 5 cm H2O between the interior of the inflatable cuff and the gas container results in the gas flow at a slow rate of less than 0.05 cc per second.

61. The apparatus according to claim 60, wherein the cuff pressure stabilizer is configured such that when the inflation lumen proximal port connector is in the connected configuration, the transient pressure difference of 5 cm H2O between the interior of the inflatable cuff and the gas container results in the gas flow at a slow rate of less than 0.02 cc per second.

62. The apparatus according to claim 61, wherein the cuff pressure stabilizer is configured such that when the inflation lumen proximal port connector is in the connected configuration, the transient pressure difference of 5 cm H2O between the interior of the inflatable cuff and the gas container results in the gas flow at a slow rate of less than 0.01 cc per second.

63. The apparatus according to claim 53, wherein the cuff pressure stabilizer further comprises an indicator module which continuously displays the pressure in the inflatable cuff.
